(19) United States
(12) Patent Application Publication
(10) Pub. No.: US 2020/0080090 A1
(43) Pub. Date: Mar. 12, 2020

(54) SELF-LIMITING CAS9 CIRCUITRY FOR ENHANCED SAFETY (SLICES) PLASMID AND LENTIVIRAL SYSTEM THEREOF

(71) Applicant: ALIA THERAPEUTICS S.R.l., Trento (IT)

(72) Inventors: Anna CERESETO, TRENTO (IT); Antonio CASINI, Trento (IT); Gianluca PETRIS, SAURIS (IT)

(21) Appl. No.: 16/340,999

(22) PCT Filed: Oct. 12, 2017
(86) PCT No.: PCT/EP2017/076129

§ 371 Date: Apr. 10, 2019

(30) Foreign Application Priority Data
Oct. 12, 2016 (IT) 102016000102542

(51) Int. Cl. C12N 15/113; C12N 15/85; C12N 15/867

(52) U.S. Cl. CPC C12N 15/1137; C12N 15/8509; C12N 15/867; C12N 2310/20; C12N 2740/15041; C12N 2750/14141; C12N 2015/8518

(57) ABSTRACT

The present invention describes a Self-Limiting Cas9 circuitry for Enhanced Safety (SLiCES) which consists of an expression unit for the Streptococcus pyogenes Cas9 (SpCas9), a first Cas9 self-targeting sgRNA and a second sgRNA targeting a chosen genomic locus. The self limiting circuit, by controlling Cas9 levels, results in increased genome editing specificity. For its in vivo utilization, SLiCES was integrated into a lentiviral delivery system (lentiSLiCES) via circuit inhibition to achieve viral particle production. Following its delivery into target cells, the lentiSLiCES circuit is switched on to edit the intended genomic locus while simultaneously stepping up its own neutralization through SpCas9 inactivation. By preserving target cells from residual nuclease activity, the present hit and go system increases safety margins for genome editing.

### FIELD OF THE INVENTION

The present invention refers to the field of biotechnology, in particular to an expression unit for CRISP/Cas9 technology and related lentiviral particles.

### STATE OF THE ART

In vivo application of the CRISPR/Cas9 technology is still limited by unwanted Cas9 genomic cleavages. Long term expression of Cas9 increases the number of genomic loci non-specifically cleaved by the nuclease.

Genome editing through the CRISPR/Cas9 technology has tremendous potential for both basic and clinical applications due to its simplicity, target design plasticity and multiplex targeting capacity. The main limit in CRISPR/Cas9 utilization are the mutations induced at sites that differ from the intended target. This is critically important for in vivo applications as unwanted alterations could lead to unfavorable clinical outcomes.

An important factor influencing the number of off-target modifications is the amount and persistence of SpCas9 expression in target cells: high concentrations of the nuclease are reported to increase off-site cleavage, whereas lowering the amounts of SpCas9 increases specificity. Transient SpCas9 expression is indeed sufficient to permanently modify the target genomic locus with decreased off-target activity as demonstrated by the enhanced specificity obtained through direct delivery of recombinant SpCas9-sgRNA complexes into target cells (Kim, S., et al., Genome Res. 2014, 24, 1012-1019; Ramakrishna, S. et al., Genome Res. 2014, 24, 1020-1027; Zuris, J. A. et al., Nat. Biotechnol. 2015, 33, 73-80).or by using a SpCas9 variant activated by inteins (Davis, K. M., et al., Nat. Chem. Biol. 2015, 11, 316-318). It is likely that any Cas9 protein present after the target locus has been edited has a substantial probability to modify additional sites. Even though direct delivery of SpCas9-sgRNA ribonucleoprotein complexes may decrease off-target effects, it is highly inefficient and unsuitable for in vivo approaches.

Slaymaker, I. M. et al. (Science 2016, 351, 84-88) describes rationally engineered Cas9 nucleases with improved specificity.

Although viral vectors are optimal delivery tools, they generate stable expression of the transferred factors which is not necessarily beneficial for CRISPR/Cas9 applications. It is known that the amount and the persistence of Cas9 result in off-target accumulation and that Cas9 permanently delivered through a lentiviral system results in consistent temporal increase of indels at off-target sites.

Approaches aimed at controlling Cas9 activity have been recently developed by exploiting various inducible systems (Nunez, J. K., et al., ACS Chem. Biol. 2016, 11, 681-688). Nevertheless, the approaches reported so far suffer of a number of limitations spanning from decreasing editing activity generated by nuclease splitting (Wright, A. V. et al., Proc. Natl. Acad. 2015, 291 Sci. U.S.A. 112, 2984-2989) or chemical modification (Davis, K. M., et al., Nat. Chem. Biol. 2015, 11, 316-318) to background activity (Nihongaki, Y., et al., Nat. Biotechnol. 2015, 33, 755-760) or extended time of required induction (Zetsche, B., et al., Nat. Biotechnol. 2015, 33, 139-142).

Kiani S., et al. (Nat Methods. 2015, 12(11): 1051-1054) disclosed that by altering the length of Cas9-associated guide RNA (gRNA) it is possible to control Cas9 nuclease activity and simultaneously perform genome editing and transcriptional regulation with a single Cas9 protein.

WO2015/070083 describes gRNA molecules (anti-Cas gRNA) that target a nucleic acid sequence that encodes the Cas9 molecule. Described are also nucleic acids comprising: a) a first nucleic acid sequence that encodes a governing gRNA molecule; and b) a second nucleic acid sequence that encodes a Cas9 molecule; wherein the governing gRNA molecule comprises a Cas9 molecule-targeting gRNA molecule (anti-Cas gRNA).

Preserving target cells from residual Cas9 activity is becoming an urgent requirement to improve the safety margins of the CRISPR/Cas9 technology towards its implementation in in vivo studies.

Aim of the present invention is to provide nucleotide sequences for downregulating Cas9 expression. Further aim of the invention is to provide an expression unit for CRISP/Cas9 technology wherein Cas9 expression is inactivated after the genome editing. Another aim of the present invention is to provide a lentiviral system for CRISP/Cas9 technology wherein achieves the efficiency of viral based delivery and simultaneously limits the amount of SpCas9 post transduction and viral integration. Aim of the present invention is to provide a method to prevent functional Cas9 or g RNA expression in bacteria and/or in packaging cells. Aim of the present invention is also providing a method for producing fully functional viral delivery of CRISP/Cas9 technology with limited amount of SpCas9 post transduction.

### SUMMARY OF THE INVENTION

Described herein is a new technology that allows genome editing through a “hit and go” Cas9 approach, which we named Self-Limiting Cas9 circuitry for Enhanced Safety (SLiCES).

Subject of the present invention is a CRISPR/CAS9 Self-Limiting Cas9 circuitry for Enhanced Safety (SLiCES) plasmid comprising:
an expression cassette for a Cas9 molecule;
a first nucleotide sequence that encodes for a sgRNA targeting the Cas9 molecule (anti-Cas9 sgRNA); and
a second nucleotide sequence that encodes for sgRNA targeting a chosen genomic locus (target sgRNA);

wherein

at least one intron is present into the open reading frame (ORF) of the expression cassette for said Cas9 molecule to form an expression cassette divided in two or more exons, and/or at least one intron is present into the nucleotides sequence encoding for the mature transcript of said anti-Cas9 sgRNA being said intron into the transcribed sequence encoding an expression cassette divided in two or more exons; and/or

the expression cassette for the Cas9 molecule and/or the sequence encoding for anti-Cas9 sgRNA is preceded by a sequence including an inducible promoter. Subject-matter of the present invention is also a viral or artificial delivery system comprising the plasmid as described above.

Further subject matter of the invention is the plasmid or the viral or artificial system as described above for use as a medicament, in particular in gene therapy. Further subject matter of the invention is also the use in vitro of the plasmid or the viral or artificial system as described above in genome engineering, cell engineering, protein expression or biotechnology.

Subject-matter of the invention is also a pharmaceutical composition comprising the plasmid, or the viral or artificial system as above describe and at least another pharmaceutically acceptable ingredient.

Further subject-matter of the present invention is also a process for preparing the viral system as above described, the process comprising
transforming a bacterium with the plasmid as above described, said bacterium wherein the expression of Cas9 and/or sgRNA is prevented by the presence of the intron or by the expression of a repressor specific for the inducible promoter or by another system apt to prevent Cas9 and/or sgRNA expression; and/or
transfecting a cell with the plasmid as above described, said cell expressing a repressor specific for the inducible promoter or said cell comprising a system for regulating Cas9 and/or anti-Cas9 gRNA expression, said cell preferably transfected with plasmids to produce a viral vector, preferably a lentiviral vector (i. e. ΔR8.9, pCMV-VSV-G).

The major advantage of SLiCES is the transient nature of Cas9 that prevents the continuous nuclease activity beyond completion of DNA target modification. In addition, SLiCES offers a variety of advantages:
Limited off-target activity;
Efficient delivery through viral systems, in particular lentiviral systems (lentiSLiCES);
Adaptability to diverse RNA guided nucleases.
Adaptability to diverse viral vectors.

Surprisingly the self limiting circuit by controlling Cas9 levels results in increased genome editing specificity. For its in vivo utilization, integration of SLiCES into a lentiviral delivery system (lentiSLiCES) via circuit inhibition to achieve viral particle production was successful. Following its delivery into target cells, the lentiSLiCES circuit is switched on to edit the intended genomic locus while simultaneously stepping up its own neutralization through SpCas9 inactivation. By preserving target cells from residual nuclease activity, our hit and go system increases safety margins for genome editing.

Overall, the “hit and go” nature of SLiCES and its adaptability to new emerging Cas9 techniques, combined with the implementation of viral delivery, allows more controllable genome editing procedures with limited unwanted off-target activity of Cas9.

Further subject-matter of present invention is a method for preventing the mature expression of a toxic transcript in a bacterium, said method comprising introducing at least one intron in the nucleotide sequence encoding for said toxic transcript; being said intron into the transcribed sequence encoding an expression cassette divided in two or more exons. Preferably the toxic transcript functions as a guide RNA, or part of it, for a nuclease; preferably the nuclease is Cas9.

### DETAILED DESCRIPTION OF THE INVENTION

The plasmid according to the invention, preferably comprises at least an intron; and a sequence encoding for an inducible promoter. More preferably, the intron is into the open reading frame (ORF) of the expression cassette for the Cas9 molecule to form an expression cassette divided in two or more exons. Most preferably the intron is only one.

The plasmid according to the invention, more preferably, is that wherein the expression cassette for the Cas9 molecule and the sequence encoding for anti-cas9 sgRNA are both preceded by a sequence including an inducible promoter. Preferably gRNA is expressed by a Pol-III recognized promoter. Preferably gRNA is expressed by U6 or H1 promoter. Preferably sgRNA is expressed by human U6 or H1 promoter. gRNA can be expressed by a tRNA promoter (Mefferd A L et al., 2015, RNA, 21, 1683-9). gRNA can be expressed by a Pol-11 promoter (Nissim L et al., 2014 Mol Cell, 54, 698-710). sgRNA can be processed by eso- or endo-RNAse (i.e. Csy4).

According to the present invention Cas9 molecules of a variety of species can be used in the methods and compositions described herein. While the much of the description herein uses S. pyogenes and S. thermophilus Cas9 molecules, Cas9 molecules from the other species can replace them, e.g., Staphylococcus aureus and Neisseria meningitidis Cas9 molecules. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

A Cas9 molecule, as that term is used herein, refers to a molecule that can interact with a gRNA molecule and, in concert with the gRNA molecule, localize (e.g., target or home) to a site which comprises a target domain and PAM sequence. The Cas9 molecule is capable of cleaving a target nucleic acid molecule.

Exemplary naturally occurring Cas9 molecules are described in Chylinski et al, RNA Biology 2013; 10:5, 727-737.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain ClipI 1262), Enterococcus italicus (e.g., strain DSM 15952), or Enterococcus faecium (e.g., strain 1,231,408). Additional exemplary Cas9 molecules are a Cas9 molecule of Neisseria meningitidis (Hou et al. PNAS Early Edition 2013, 1-6) and a S. aureus Cas9 molecule.

In an embodiment, a Cas9 molecule, comprises an amino acid sequence: having 60%, 65%, 70%, 75%, 80%, 85%, 90%, 95%, 96%, 97%, 98%, or 99% homology with or is identical to any Cas9 molecule sequence described herein or a naturally occurring Cas9 molecule sequence, e.g., a Cas9 molecule from a species listed herein or described in Chylinski et al., RNA Biology 2013, 10:5, 727-737; Hou et al. PNAS Early Edition 2013, 1-6.

Cas9 can also be an engineered Cas9 molecule as recently developed (Kleinstiver B. P., et al., Nature. 2016. 529, 490-5; Slaymaker I. M., et al., Science. 2016. 351, 84-8. Nuñez J. K., et al., ACS Chem. Biol. 2016. 11, 681-688; Wright A. V., et al. Proc. Natl. Acad. Sci. U.S.A. 2015. 112, 2984-2989. Nihongaki Y., et al., Nat. Biotechnol. 2015. 33, 755-760. Zetsche, B., et al., Nat. Biotechnol. 2015.33, 139-142).

Cas9 molecule can also be mutated or engineered to be a nickase (e.g. D10A, D10A/D839A/H840A and D10A/D839A/H840A/N863A mutant domains), a mutant of Cas9 nuclease domains unable to cleave the DNA (e.g. D10A, D10A/D839A/H840A, and D10A/D839A/H840A/N863A mutant domains), a fusion with a nuclease domain (e.g. Fok-I) or a nucleic acid-editing domain (e.g. DNA deaminase).

Cas9 molecule can be substituted by different subtype and class of RNA guided nucleases like AsCpf1 and LbCpf1 examples are included in Shmakov S., et al., Mol Cell 2015, 60, 385-397.

RNA guided nucleases can be substituted by DNA guided nucleases (e.g. Natronobacterium gregory Argonaute), recently described for genome editing in Gao F., Nature Biotechnology. 2016. 34, 768-773.

According to the invention the sgRNA can target any DNA sequence known in the art; the targeting sgRNA can be modified to have different affinity to Cas9 molecule; the targeting sgRNA can be modified to be more stable in targeted cells; the targeting sgRNA can be chemically modified (i.e. phosphorothioate RNA, RNA deamination); the targeting sgRNA can be fused to additional RNA domains at it 5′ and 3′ ends (i.e. MS2 repeats, RNA hairpins); sgRNAs are not limited to a single molecule but can be a gRNA which can be form by different RNAs molecules similarly to crRNA and tracr-RNA; particularly preferred is a target sgRNA that targets a therapeutically interesting locus. Target sgRNA loci are genes or intergenic sequences having effects on somatic, stem or cancer cell growth or fitness, either as single target or in combination (i.e. synthetic lethality). Target sgRNA can encode genes involved cell methabolism. Target sgRNA loci can encode for essential genes for virus infection persistence or replication. Particularly preferred loci can be, for example, HBG1, HBG2, HBB, Prp, HTT, PCSK9, SERPINA1, LEDGF/p75; CCRS, CXCR4, TCR, BCR, VEGFA, ZSCAN, EMX1, ROSA26, AAV1, β-globin, CFTR.

The target of sgRNA can be a DNA sequence of viral origin (van Diemen F. R. et al., PLoS Pathog. 2016. 12(6):e1005701; Seeger C and Sohn J. A Molecular Therapy—Nucleic Acids (2014) 3, e216). Application of SLiCES can be intended to clear virus from infected cells by targeting viral genetic elements important for virus fitness and replication. Particular preferred viral loci can be for example:
HIV-1 genome, preferentially conserved regions, LTR, protease, integrase, Gag and GagPol;
retrovirus genome, preferentially conserved regions, LTR, protease, integrase, Gag and GagPol;
HBV genome, preferentially conserved regions, RT, surface Ag, core genes;
Herpes simplex virus (HSV) genome, preferentially conserved regions;
Human cytomegalovirus (HCMV) genome, preferentially conserved regions;
Epstein-Barr virus (EBV) genome, preferentially conserved regions;
Human Papillomavirus genome and episomes, preferentially E6 or E7.

Application of SLiCES on viral genetic elements can be intended to facilitate engineering of recombinant viruses (Suenaga T et al., Microbiol Immunol. 2014. 58, 513-22; Bi Y et al., LoS Pathog 10(5):e1004090).

Viral delivery system comprising the SLiCES plasmid of the invention can be DNA or RNA viruses, preferentially lentivirus, retrovirus, SIV, EIAV, AAV, Adenovirus or Herpervirus. Preferably according to the invention the viral delivery system is a lentiviral system (lentiSLiCES). Herein after is reported an example of lentiviral system comprising the SLiCES plasmid of the invention; for retrovirus, SIV, EIAV the system can be photocopied identical; for AAV, Adenovirus or Herpervirus the system can be adapted based on the same principle.

For an aspect the present invention relates to a genetically-modified micro-organism, preferably a bacterium, comprising the plasmid as above described. For an aspect the present invention relates to a cell, preferably a mammalian cell, transfected with the plasmid as above described.

A bacteriophage can encodes its own CRISPR/Cas system (Seed K D et al., Nature. 2013. 28, 489-91; Bellas C et al., Frontiers in Microbiology 2015. 6, 656). Bacteriophage could be simply engineered to be a viral delivery system for SLiCES to control timing and increase specificity of targeted double strand breaks formation in a specific bacterial population. The SLiCES system delivered by bacteriophages can be used for example to change composition of a heterogeneous bacterial population, or to remove specific phages form a bacterial population. To function against bacteriophage the SLiCES system could not contain eukaryotic introns since the SLiCES should be fully functional in bacteria that are not able process them.

Bacterial cells could be used to deliver the SLiCES circuit to other bacterial cells (i.e. bacterial conjugation delivering SLiCES DNA, RNA or protein) or to mammalian cells (infection of mammalian cells by engineered Trypanosoma cruzi, Plasmodium Falciparum containing SLiCES circuit and delivering SLiCES DNA, RNA or protein). Artificial delivery system comprising the SLiCES plasmid of the invention can be for example organic or inorganic vehicles (artificial or ghost cells, liposomes, vesicles, exosomes, bacterial outer membrane vescicles, fatty acid droplets, proteins, peptides, synthesis compounds, metallic and non-metallic particles and nanoparticles, fullerene, carbon nanotubes), mechanic devices (microfluidic squeezing, microinjection, nanomachines, micromachines), hydrodynamic injection, electroporation.

Consequently the plasmid, viral and artificial system of the invention can result useful in the treatment of several monogenic disorders where genome editing could be used full such as: Cystic fibrosis, SCID (Severe combined immunodeficiency syndromes), Wiskott-Aldrich syndrome, Haemophilia A and B, Hurler syndrome, Hunter syndrome, Gaucher disease, Huntington's chorea, Duchenne muscular dystrophy, Spinal Muscular Atrophy, Canavan disease, Chronic granulomatous disease, Familial hypercolesterolaemia, Fanconi's anemia, Purine nucleoside phosphorylase deficiency, Ornithine transcarbamylase deficiency, Leucocyte adherence deficiency, Gyrate atrophy, Fabry disease, Pompe disease, Tay sachs disease, Nieman-Pick A, B, Sly syndrome, Sanfilippo disease, Maroteaux-Lamy disease, Aspartylglucosaminuria disease, Amyotrophic lateral sclerosis, Junctional epidermolysis bullosa, Leukocyte adhesion disorder, Farber disease, Krabbe disease, Wolman disease.

Other disease potentially benefit from the invention could be but are not limited to: high cholesterol, antitrypsin deficiency, cancer, diabetes, infective bacterial and viral diseases. Some examples are included in Maeder M. L and Gersbach C. A. Molecular Therapy 2016, 24, 430-446.

SLiCES limits Cas9 re-cleavage of a target DNA locus after HDR (homologous directed repair), increasing probability of accurate HDR. Accurate HDR is essential in most genome editing application in cell biology and molecular medicine. Complicated and time consuming procedure have been developed to address this issue (Paquet D. et al., Nature 2016, 533, 125-9). SLiCES and lentiSLiCES can be delivered together with a donor DNA to induce HDR. The Cas9 self-inactivation prevent further cleavage of the genetic corrected loci without requiring additional protective mutations currently required to prevent Cas9 re-cleavage. A protective mutation is a mispairing between donor DNA and targeted locus, which is located within gRNA targeting sequence or within Cas9 recognized PAM sequence.

Insertions of protective mutations should be avoided or limited as may unpredictably affect correct function of a target locus.

Genome-wide gene knockout screening, using for instance the Brunello, Brie and GeCKO libraries, could take advantage of SLiCES and lenti-SLiCES to reduce off-targets effects which can affect accuracy and reproducibility of the screen.

According to the invention the anti-Cas9 sgRNA can be any sgRNA capable of targeting a Cas9 molecule; for example an anti-Cas9 sgRNA as disclosed in WO2015/070083. Preferably according to the invention anti-Cas9 sgRNA are those encoded by a sequence of 17-23 nucleotides, preferably starting with G. Preferably anti-Cas9 sgRNA encoding sequence is a sequence having at least a 60% homology with a sequence selected in the group consisting of SEQ ID N. 1-6. More preferably anti-Cas9 sgRNA encoding sequence is a sequence having at least a 70%, 80%, 90%, 100% homology with a sequence selected in the group consisting of SEQ ID N. 1-6. Most preferably anti-Cas9 sgRNA encoding sequence is a sequence identical to a sequence selected in the group consisting of SEQ ID N. 1-3 for targeting a Cas9 molecule of S. pyogenes or is a sequence identical to a sequence selected in the group consisting of SEQ ID N. 4-6 for targeting a Cas9 molecule of S. thermophilus.

Preferably the plasmid according to the invention comprises, subsequent and adjacent to the sequence encoding for the anti-Cas9 sgRNA and/or the target sgRNA is present a sequence encoding for a gRNA backbone or encoding for an optimized gRNA backbone. Preferably the sequence encoding for the anti-Cas9 sgRNA is adjacent to a sequence encoding for an optimized gRNA backbone and the sequence encoding for the target sgRNA is followed by a sequence encoding for a gRNA backbone. Preferably the sequence encoding for a gRNA backbone is SEQ ID N.7 and the sequence encoding for an optimized gRNA backbone is SEQ ID N. 8.

To avoid the leaky expression of SpCas9, and the consequent degradation of DNA during plasmid preparation in bacteria, an intron was introduced into the SpCas9 open reading frame to form an expression cassette divided in two exons (exon 1 and 2, schematized in FIG. 8). As splicing does not occur in bacteria, the transcripts produced are translated in bacteria as a catalytically inactive SpCas9 fragment. As intron can be introduced any nucleotide sequence that is removed by RNA splicing during maturation of the final RNA product. Suitable are in example:
nuclear pre-mRNA introns (spliceosomal introns), which are characterized by specific intron sequences located at the boundaries between introns and exon (5′ splice site, branch point, polypyrimidine tract, 3′ splice site);
introns in transfer RNA genes that are removed by proteins (tRNA introns);
self-splicing introns that are removed by RNA catalysis.
RNA ribozymes.

Preferably the intron is derived from the mouse immunoglobulin heavy chain precursor V-region intron. More preferably the intron identical to SEQ ID N.9. A preferential intron is also rabbit β-globin intron 2.

Most different introns present in eukaryotic genes could be used to prevent Cas9 expression in bacteria and some of them could be exploited also to restrict Cas9 expression to specific eukaryotic tissues.

An intron could be used also to prevent correct expression of gRNA, in particular self targeting gRNA, in bacteria. The intron could be introduced into variable or constant parts of gRNA. Similarly an intron could be introduced into cr- or tracr-RNAs.

To prevent leaky Cas9 expression in bacteria its expression could be regulated by an inducible promoter in place of an intron within Cas9 gene. This could prevent Cas9 expression while DNA plasmid is amplified (es. DH5alfa-Z1, carrying Lac Repressor and Tet Repressor encoding genes driven by the constitutive promoters Placiq and PN25, respectively); see also below for inducible promoters.

Similarly inducible promoters could be used to prevent gRNA expression, in particular self targeting gRNA, in bacteria (for example H1-TetO promoter).

To prevent leaky Cas9 expression in bacteria riboswitches (RNA elements in mRNA that control gene expression in cis in response to their specific ligands) could be used to drive Cas9 translation and could also be used in place of an intron within Cas9 gene. Both naturally regulated and artificial riboswitches and IRES, preferentially if controlled by ligands (i.e. theophylline, Flavin MonoNucleotide, tetracycline, doxycycline and sulforhodamine B) could be used to prevent Cas9 expression in bacteria.

The Cas9 and gRNA expression in bacteria could also be controlled by use of antisense nucleotides acting on RNA or DNA encoding the Cas9 gene and gRNA. To circumvent the self-cleavage activity during lentiviral vector production, inducible promoters were introduced to regulate preferably both Cas9 and the anti-Cas9 sgRNAs expression. The inducible promoter is preferably selected in the group consisting of Tetracycline inducible (TetO) promoters, AAREs (amino acid response elements), LacO promoter, LexA promoter, heat shock promoter, light inducible promoter, ecdysone responsive promoter.

The inducible promoter is negatively regulated by a specific corresponding repressor, which is expressed in producing cells. The TetO promoter is negatively regulated by a specific repressor, TetR, which is expressed in producing cells and, in the absence of doxycycline, inhibits transcription through binding to tetracycline operator sequences located within the promoter region (schematized in FIG. 8b). AAREs (amino acid response elements) expression system, rapidly activated by diet deficient of one EAA (essential amino acid); packaging cells must be grown in presence of EAA to prevent expression (Chaveroux C., et al., Nat Biotech. 2016. 34, 746-751). LacO promoter, negatively regulated by Lacl repressor; inhibit transcription in the absence of IPTG (Isopropyl β-D-1-thiogalactopyranoside). LexA operator, LexA repressor; inhibition of transcription unless RecA and DNA damage are present. Heat shock promoters, are repressed unless “high” temperature. Light inducible promoters. Muristerone A and ponasterone A, analogs of ecdysone receptor and an ecdysone responsive promoter, driving the expression of the gene of interest.

As described for bacteria also in packaging cells to circumvent the self-cleavage activity during lentiviral vector production a riboswitch or an inducible IRES could be introduced to regulate preferably Cas9 or gRNA expression.

Similarly antisense nucleotides could be used to regulate Cas9 and gRNA expression in packaging cells to circumvent the self-cleavage activity during lentiviral vector production.

The described methods to prevent gRNA expression could be extended on associated non-self targeting gRNAs, preferentially if their expression would be toxic/detrimental and decreasing efficiency or safety of lenti-SLiCES vectors preparation.

The plasmid of the invention can further comprise a nucleotide sequence useful for the selection or isolation of the viral particle or of the transduced cells or having an additional effect on tranduced cells as for example containing one or more:

IRES (internal ribosome entry site, preferentially of viral origin like ECMV-IRES, or a non viral IRES, of particular interest are the tissue specific IRES like FGF-2 IRES, or an artificial IRES and riboswitches preferentially in controlled by ligands i.e. theophylline, Flavin MonoNucleotide, tetracycline, doxycycline and sulforhodamine B) near to its regulated gene, i.e. blasticidin resistance gene,
reporter gene (GFP, Luciferase, beta-Galattosidase),
protein fusions with engineered amino acidic tags, biotin acceptor tags), gene useful to control grow and survival of targeted cells (es. Thymidine Kinase),
gene expressing a therapeutic protein, which can enhance survival/fitness of transduced and non-transduced cells or have a biological effect (i.e. control of immune response, metabolic effect, vascular remodeling) on targeted or non-targeted cells (IL-2, IL-8, GM-CSF, insulin, VEGFA).

According to an embodiment of the invention, the plasmid of the invention comprises a 5′LTR, 3′LTR-SIN, hU6 promoter, gRNA backbone, target sgRNA, hH1TetO promoter, anti-cas9 sgRNA sequence, optimized gRNA backbone, CMV-TetO promoter, FLAG-NLSSpCas9-NLS, intron, ECMV-IRES, blasticidin resistance gene, WPRE. Such a sequence is exemplified by SEQ ID N. 10.

To further improve the SLiCES strategy, Integrase Defective Lentiviral Vectors (IDLV) could be used to maintain the viral-based efficiency in cellular delivery, while enhancing the transient peak-like nature of Cas9 expression.

To transfer SLiCES into a retroviral vector is sufficient to transfer the transgenes present in the Lenti SLiCES to the retroviral transfer vector.

To transfer SLiCES into a EIAV vector is sufficient to transfer the transgenes present in the Lenti SLiCES to the EIAV transfer vector.

To transfer SLiCES into a SIV vector is sufficient to transfer the transgenes present in the Lenti SLiCES to the SIV transfer vector.

To transfer SLiCES into an AAV vector could be used a small nucleotide size Cas9, like SaCas9, including an intron within its gene and having inducible promoter on Cas9 and/or on self targeting gRNA.

To transfer SLiCES into Adenoviral vector is sufficient to transfer the transgenes present in the SLiCES to the Adenoviral vectors using standard recombination or cloning techniques to create replication competent, replication defective and helper dependent vectors.

To transfer SLiCES into a Herpes vector is sufficient to transfer the transgenes present in the SLiCES to the Herpes vectors using standard techniques for transgene insertion.

For an aspect of the present invention, subject-matter is also an anti-Cas9 sgRNA encoded by a nucleic acid sequence selected in the group consisting of SEQ ID n.1-6.

Packaging cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells. Particular cells include mammalian cells.

Preferably packaging cells to produce Lenti-SLiCES are mammalian cell, in particular HEK-293 cells, which could be modified to express a repressor to prevent spurious activation of SLiCES activity. Vice versa packaging cells could be engineered to lack a gene required for SLiCES activation.

Packaging cells could also be artificial or in vitro systems for RNA protein expression.

For an aspect the present invention relates to a method for detecting DNA breaks, preferentially Cas9 off-targets, in in vitro cultured cells or in in vivo animal models, said method comprising using the plasmid as above described wherein the plasmid is introduced directly or in the form of a non-integrating vector, such as IDLV and AAV. In said method the plasmid or the non-integrating viral vectors are cleaved by activation of the SLiCES activity. As a result the SLiCES plasmids or vectors are captured into genomic DNA breaks by the DNA repair machinery and are thus integrated into the genome. By amplifying the loci of integration is possible to detect DNA fragile sites and in cells treated with a nuclease, such as Cas9, detect on-target and off-target cleavage sites.

### BRIEF DESCRIPTION OF THE FIGURES

FIG. 1. Long term expression of Cas9 delivered through a lentiviral vector correlates with the accumulation of off-target cleavages. (a) Time course curves of the percentages of 293-iEGFP non-fluorescent cells obtained by the transduction with a lentiviral vector (lentiCRISPR) expressing SpCas9 together with either a perfectly matching sgRNA (sgGFP-W) or two different sgRNAs containing one or two mismatches with the target sequence (sgGFP-M and -MM, respectively). A vector expressing an irrelevant sgRNA was used as control (sgCtr). (b) As in (a) using a lentiviral vector expressing a SpCas9 variant with increased fidelity (eSpCas9(1.1)). (c) DNA modification specificity, defined as on-target/off-target indels frequency ratio, after long term SpCas9 expression with sgRNAs targeting the VEGFA and ZSCAN endogenous loci. Percent modification of previously validated off-target sites was quantified by TIDE analysis after one week and 21 days post-transduction. For all the experiments, cells were selected with puromycin in order to eliminate the non-transduced cells. In panels (a-c) data presented as mean±s.e.m. for n=2 independent experiments.

FIG. 2. The SLiCES circuit. (a) Scheme of the SLiCES circuit. SpCas9 is expressed together with sgRNAs directed to its own open reading frame (ORF) for self-limiting activity and to a selected target sequence. (b) Regulation of SpCas9 and EGFP target gene expression by the SLiCES circuit. Western blot analysis of 293T cells co-transfected with plasmids expressing EGFP, SpCas9 and sgRNAs fully (sgGFP-W) or partially matching (sgGFP-M) the EGFP coding sequence in combination with three sgRNAs targeting the SpCas9 ORF (sgCas-a, -b, -c) or a control sgRNA (sgCtr), as indicated. Lane (-) corresponds to a reference sample containing the non-targeting sgCtr only. Transfection efficiency was normalized using roTag tagged MHC-la expression plasmid (Transf-ctr). SpCas9 was detected using an anti-FLAG antibody. Lower graph reports the ratio of the percentages of decreased EGFP levels obtained using sgGFP-W (on-target) over the percentages obtained with sgGFP-M (off-target) in the presence of sgCas-a, -b, -c as indicated. (c) Target specificity of SpCas9 activity using different SLiCES circuits. On/off ratios were obtained from the percentage of EGFP negative cells after targeting a single chromosomal EGFP gene copy (293-iEGFP cells) with sgGFP-W (on-target) relative to sgGFP-M (off-target) in combination with different SLiCES circuits (sgCas-a, -b or -c) or a non-targeting (sgCtr) sgRNA, as indicated in the graph. (d) Target specificity of SpCas9 activity expressed as on/off ratios as in (c) using optimized sgRNAs, as indicated in the graph. (e) Target specificity of SpCas9 activity expressed as on/off ratios using different self-limiting circuits applied to a gene substitution model. On/off ratios were obtained from the percentage of EGFP positive cells generated by SpCas9 homology-directed repair of the EGFP-Y66S mutation with the sgGFP-M (on-target) relative to the sgGFP-W (off-target) sgRNAs in combination with a DNA donor plasmid (carrying wild-type EGFP sequence) in 293-iY66S cells containing a single mutated EGFP gene copy. (f) Indels formation induced by the SLiCES circuit (sgCas-a-opt) targeting the VEGFA, ZSCAN2, EMX1 loci and their respective validated off-target sites. Fold increase (F.I.) of the on/off ratio with the sgCasa-opt relative to the sgCtr is reported below the graphs for each off-target. Percent modification was quantified by TIDE analysis. Error bars represent s.e.m. for n≥2.

FIG. 3. Regulation of SpCas9 and EGFP-Y66S expression by the SLiCES circuit. Western blot of cells co-transfected with plasmids expressing EGFP-Y66S, SpCas9, sgRNAs perfectly matching (sgGFP-M) or containing one mismatch (sgGFP-W) with the EGFP-Y66S target sequence together with sgRNAs specific for the SpCas9 ORF (sgCas-a, -b, -c) or a control sgRNA (sgCtr), as indicated. Lane (-) corresponds to a reference sample containing the non-targeting sgCtr only. Transfection efficiency was normalized using roTag tagged MHC-la expression plasmid (Transf-ctr). SpCas9 was detected using an anti-FLAG antibody. Lower graph reports the ratio of the percentages of decreased EGFP-Y66S levels obtained using sgGFP-M (on-target) over the percentages obtained with sgGFP-W (off-target) in the presence of sgCas-a, -b, -c, as indicated.

FIG. 4. EGFP disruption by SLiCES circuits. (a) Percentage of nonfluorescent 293-iEGFP cells obtained after expression of different self-limiting SpCas9 circuits. Cells were transfected with sgRNAs perfectly matching (sgGFP-W) or containing one mismatch (sgGFP-M) with the EGFP ORF together with three sgRNAs targeting the SpCas9 ORF (sgCas-a, -b, -c) or a control sgRNA (sgCtr), as indicated. The dashed line represents the average background of EGFP negative cells. Error bars represent s.e.m. for n=2. Data presented as mean±s.e.m. for n=2 independent experiments. (b) Representative T7 Endonuclease assay from cells expressing different SLiCES circuits. The on/off specificity ratio was calculated by measuring indels formation in the EGFP gene in the presence of sgGFP-W or sgGFP-M together with a control sgRNA or the three sgRNAs targeting the SpCas9 ORF (sgCas-a, -b, -c). Lane (-) corresponds to a reference sample containing the non-targeting sgCtr only. (*) Indicates the expected band obtained by T7 endonuclease activity.

FIG. 5. Effect of sgRNAs optimization on SLiCES circuit. (a) Percentage of non-fluorescent 293-iEGFP cells obtained after transfection of SpCas9 with sgRNAs targeting EGFP (sgGFP-W or sgGFP-W-opt, if optimized) or containing a single mismatch (sgGFP-M or sgGFP-M-opt, if optimized) together with the sgCas-a. The optimized version of the SLiCES sgRNA (sgCas-a-opt) was tested with both standard and optimized sgRNAs targeting EGFP, as indicated. Data presented as mean±s.e.m. for n=2 independent experiments. (b) Percentage of non-fluorescent 293-iEGFP cells obtained after transfection of SpCas9 with sgRNAs targeting EGFP (sgGFP-W) or containing a single mismatch (sgGFP-M) together with the sgCas-c or sgCas-c-opt, if optimized. data presented as mean±s.e.m. for n=2 independent experiments. (c) Western blot analysis of 293T cells co-transfected with SpCas9 and sgCas9-a or sgCas-a-opt and sgCas9-c or sgCas-c-opt. SpCas9 was detected using an anti-FLAG antibody. Transfection efficiency was normalized using roTag tagged MHC-la expression plasmid (Transf-ctr).

FIG. 6. Specificity of homology-directed repair mediated by SLiCES. Percentage of fluorescent 293-iY66S cells obtained after transfection with a donor DNA plasmid (carrying a non-fluorescent fragment of wt-EGFP), SpCas9 together with sgRNAs matching (sgGFP-M) or containing one mismatch with the EGFP-Y66S target sequence (sgGFP-W) and the three sgRNAs targeting the SpCas9 ORF (sgCas-a, -b, -c or sgCas-a-opt) or a control sgRNA (sgCtr), as indicated. Data presented as mean±s.e.m. for n=2 independent experiments. Homology-directed repair in the absence of sgGFP-M or sgGFP-W was about 0.01%.

FIG. 7. Activity of SLiCES with Streptococcus thermophiles CRISPR1/Cas9. (a) Schematic representation of the SV5-GFP-based NHEJ reporter. The target sequence recognized by the sgRNA of interest is inserted between the SV5 tag and the EGFP coding sequences, with the EGFP ORF positioned out of frame with respect to the starting ATG codon for the SV5 tag ORF. A stop codon has been added to the SV5 frame, immediately after the target sequence, to stop its translation. After SpCas9-mediated cleavage of the target sequence and repair by NHEJ, indel mutations are inserted randomly at the breakpoint, allowing the shift of the EGFP ORF in the same frame of the SV5 tag ORF. The expression of the SV5-EGFP is analyzed by fluorescence detection or by western blot analysis. (b) Evaluation of St1Cas9 activity expressed through the SLiCES system. Western blot of 293T cells transfected with St1Cas9, the NHEJ reporter carrying either a target sequence that fully base pairs with the sgRep-SV5 (NHEJ-Rep.W) or including one mismatch (NHEJ-Rep.M), the sgRNA sgRep-SV5 and three different St1Cas9 targeting sgRNAs (sgCas-St1, -2, -3). St1Cas9 mediated cleavages are detected by frameshift of the EGFP ORF and SV5-EGFP expression by the NHEJ reporter as described in (a). Lane (sgCtr) corresponds to a sample transfected with a non-self-targeting sgRNAs; lane (-) corresponds to a sample transfected with a non-targeting sgRNA. St1Cas9 was detected using an anti-FLAG antibody. Western blot is representative of n=2 independent experiments. (c) Modulation of St1Cas9 expression by self-limiting circuits increases on target specificity. On/off target ratios calculated from levels of SV5-EGFP expression obtained from cells transfected with NHEJRep. W or NHEJ-Rep.M together with sgRep-SV5 in combination with St1Cas9 targeting sgRNAs (sgCas-St1, -2, -3) or a non-self-targeting sgRNAs sgCtr as in (b). Data presented as mean±s.e.m. for n=2 independent experiments.

FIG. 8. The lentiSLiCES system. (a) Graphical representation of lentiSLiCES viral vector. (b) Steps required for the production of the lentiSLiCES viral vectors. SpCas9 expression is prevented in bacterial cells to allow plasmid amplification through the introduction of a mammalian intron within the SpCas9 open reading frame. Production of lentiSLiCES viral particles is obtained in cells stably expressing the Tetracycline Repressor (TetR) to prevent SpCas9 and sgCas self-limiting sgRNA expression driven by Tet repressible promoters. In target cells the absence of the TetR allows the expression of the lentiSLiCES circuit leading to target genome editing and simultaneous SpCas9 downregulation.

FIG. 9. lentiSLiCES circuit behaviour in viral vector packaging cells. Western blot analysis of 293TR cells transfected with EGFP and self-limiting or nonself-limiting transfer vectors carrying sgGFP-W (lentiSLiCES-W or lentiCtr-W, respectively) or with lentiSLiCES carrying a non-targeting sgRNA (lentiSLiCES-Ctr). Cultures were treated as indicated with doxycycline to upregulate expression of SpCas9 and of the self-targeting sgCas-a. SpCas9 was detected using an anti-FLAG antibody. Western blot is representative of n=2 independent experiments.

FIG. 10. Genome editing with lentiSLiCES vectors. (a) EGFP knock-down by lentiSLiCES vectors. Time course curves of the percentages of EGFP negative 293-multiEGFP cells, following transduction with lentiviral vector carrying self-targeting (lentiSLiCES) or non-self-targeting (lentiCtr) sgRNAs in combination with either sgGFP-W (on-target) or sgGFP-M (off-target) sgRNAs, as indicated in the graph. (b) Target specificity of SpCas9 delivered through the lentiSLiCES. On/off ratios were calculated from the percentages of EGFP negative cells reported in (a). Below the graphs is reported the fold increase (F.I.) of specificity calculated from the on/off ratios at each time point. (c) Indels formation induced by lentiSLiCES vectors at the ZSCAN and VEGFA loci and at their validated off-target sites. Percent modification was quantified by TIDE analysis on genomic DNA collected 20 days post-transduction and selection with blasticidin. Values indicate the on/off ratios calculated from indels obtained with each off target. (d) Expression levels of SpCas9 at the indicated time points after transduction with lentiSLiCES or with lentiCtr. SpCas9 was detected using an anti-FLAG antibody. (e) SpCas9 activity monitored by SV5-EGFP protein levels produced by the NHEJ-reporter plasmid transfected in 293-multiEGFP cells before or 28 days after transduction and detected at 2 days or 30 days post-transduction, as indicated, with lentiSLICES targeting (lentiSLiCES-W) or non-targeting EGFP (lentiSLiCES-Ctr). The activity of the non-self-limiting lentiCtr-W vector targeting EGFP was monitored at the same time points for comparison. Error bars represent s.e.m. for n=2.

### EXPERIMENTAL SECTION

Discussion

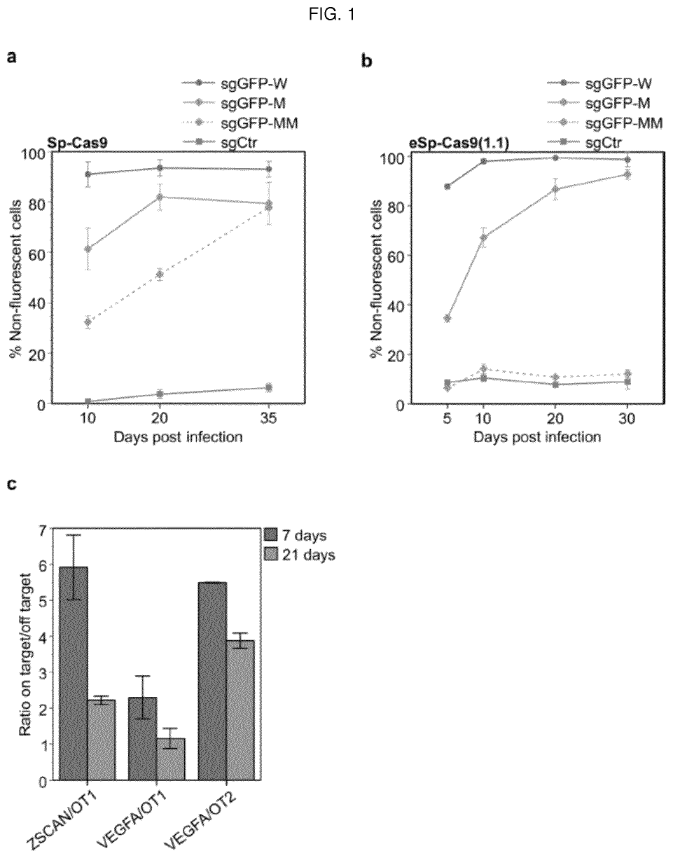

To evaluate the off-target activity produced by long term expression of SpCas9, 293-iEGFP cells were transduced carrying a single chromosomal copy of EGFP with a lentiviral vector expressing SpCas9 together with sgRNAs that can fully (sgGFP-W) or partially (sgGFP-M or sgGFP-MM) anneal to EGFP. The tolerance of SpCas9 for single (sgGFP-M) or double (sgGFP-MM) mismatches in cleaving EGFP allows for the quantification of the nuclease specificity. While the percentage of EGFP negative cells obtained with the on target sgRNA quickly reached a plateau at 10 days post-infection, the two mismatched sgRNAs generated unspecific EGFP knock-outs which accumulated over time (FIG. 1a). The delivery of the recently developed more specific eSpCas9(1.1) variant (Slaymaker, I. M. et al. Science 2016, 351, 84-88) guided by the same sgRNAs only partially reverses the time dependent accumulation of off-target cleavages (FIG. 1b). Consistently, the analysis of two genomic loci (ZSCAN and VEGFA) and related off-target sites (Kleinstiver, B. P. et al. Nature 2016, doi:10.1038/nature16526), indicated that the on/off ratios decreased over time, thus confirming increased off-target cleavages (FIG. 1c). These results clearly show that the delivery of SpCas9 through a conventional lentiviral system correlates with increased off-target activity and this is particularly evident over time due to prolonged SpCas9 expression.

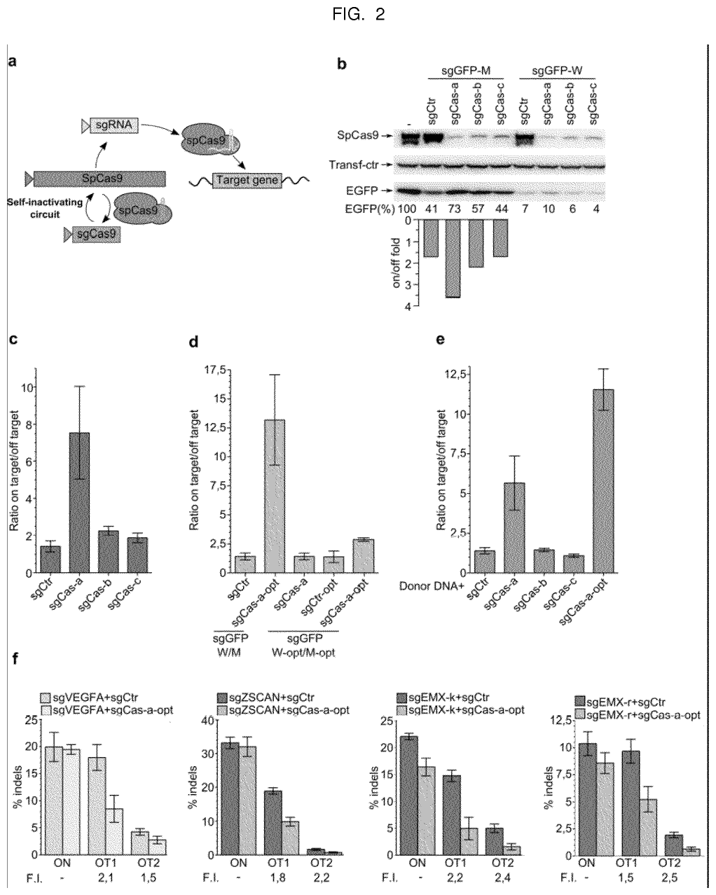

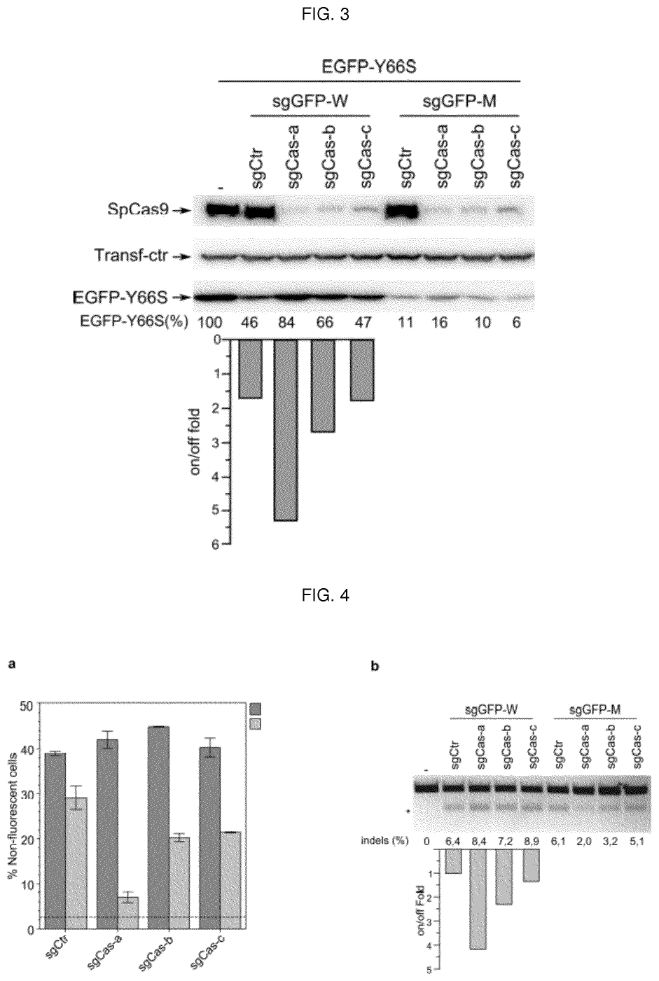

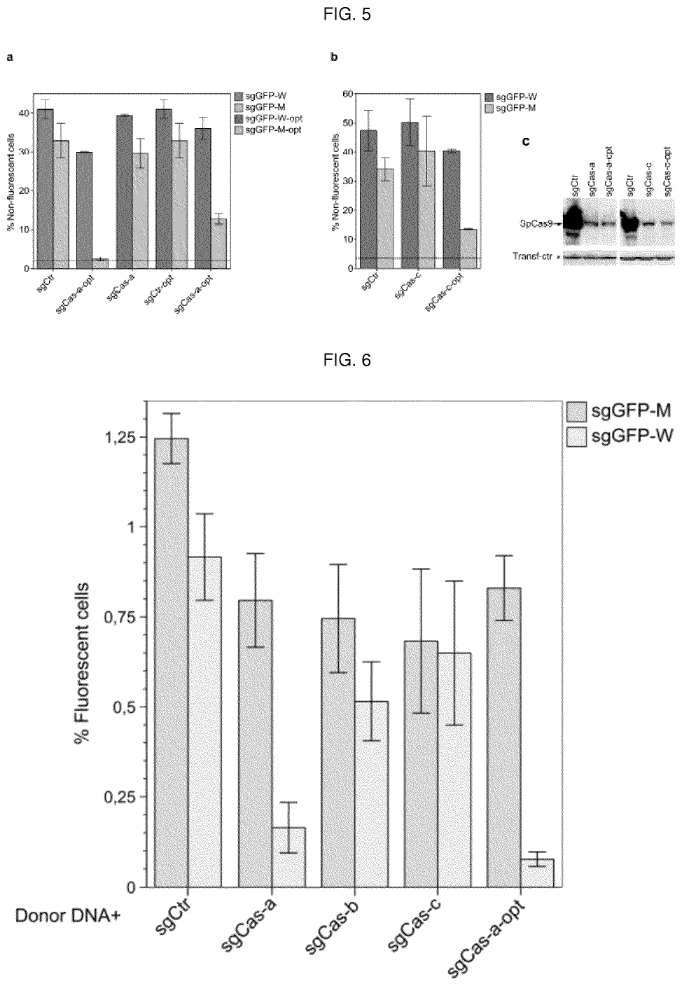

To generate a transient SpCas9 activity peak in target cells according to the present invention it was developed a Self-Limiting Cas9 circuitry for Enhanced Safety (SLiCES) (schematized in FIG. 2a). The self-limiting SpCas9 circuitry was set up in EGFP expressing cells by using three different sgRNAs targeting three regions of the SpCas9 coding sequence (sgCas-a, -b and -c) (see Supplementary Discussion below) which were shown to efficiently downregulate SpCas9 levels when co-expressed with SpCas9 (FIG. 2b, upper panel). Co-expression of any of the three self-targeting sgRNAs (sgCas-a,-b or -c) together with a sgRNA that fully base pairs with the EGFP target sequence (sgGFP-W) reduced intracellular EGFP to levels (4-10% of residual protein) similar to the EGFP content detected in cells co-transfected with the same sgGFP-W and a control sgRNA (sgCtr) (FIG. 2b). These results demonstrate that DNA editing activity is not impaired when SpCas9 is inactivated through the SLiCES circuitry. A similar experiment performed using a sgRNA targeting EGFP with a single mismatch within the seed region at the last nucleotide before the PAM (protospacer adjacent motif) sequence (sgGFPM) showed non-specific EGFP downregulation, with almost 60% decrease of EGFP intracellular levels. This effect was less pronounced (˜25-55% reduction) in cells where SpCas9 expression was downregulated through the self-limiting Cas9 circuitry (sgCas-a, -b or -c) (FIG. 2b). The different levels of non-specific EGFP downregulation closely reflected the ability of individual sgRNA to decrease the intracellular levels of SpCas9: sgCas-a, which generated the lowest non-specific EGFP downregulation (73% residual EGFP, FIG. 2b), showed the highest SpCas9 disruption activity (FIG. 2b, upper panel). Similar results were obtained with a reciprocal experiment where cells were transiently transfected with a mutated EGFP target characterized by a single nucleotide substitution (EGFP-Y66S) that fully matched the sgGFP-M sequence (FIG. 3). The improved target specificity of about 2-3 fold (FIG. 1b, lower panel) as defined by the ratio between SpCas9 activity in cells targeted by the perfectly matched sgRNA over the mismatched sgRNA carried by SLiCES, was also confirmed in 293-iEGFP cells carrying a single chromosomal copy of the EGFP gene (5-fold improvement) (FIG. 2c and FIG. 4). To test whether the optimization of the sgRNAs may further improve the on-target specificity, the sgRNAs were structurally modified to increase their transcription and interaction with SpCas9 (Chen, B. et al. Cell 2013, 155, 1479-1491). The optimization of the sgRNA targeting SpCas9, which enhances the efficiency of nuclease removal, produced a significant improvement in cleavage specificity (FIG. 2d and FIG. 5a) of about 9-fold. Consistently, the optimization of the least active self-inactivating SpCas9 sgRNA (sgCas-c) resulted in reduced off-target activity paralleled by a further decrease in SpCas9 intracellular levels (FIGS. 5b and c). Conversely, the optimization of the sgRNA towards the target site (sgGFP-W-opt and sgGFP-M-opt) did not increase specificity in combination with sgCas9-a or sgCas9-a-opt (FIG. 2d and FIG. 5a). Presumably the enhanced downregulation of EGFP driven by the sgGFP-W-opt, which also correlated with increased off-target cleavages induced by the sgGFP-M-opt sgRNA, could not be counteracted by sufficiently rapid SpCas9 downregulation mediated by both versions of the self-limiting SpCas9 sgRNA (FIG. 2d and FIG. 5a).

In conclusion, the SLiCES circuitry produced the highest on target specificity when composed of a sgRNA optimized towards SpCas9 (sgCas-a-opt) efficiently downregulating SpCas9, in combination with a non-optimized sgRNA targeting the site of interest (sgGFP-W/M). A parallel experiment aimed at validating the on-target specificity of the SpCas9 self-limiting circuitry was performed in cells carrying a single chromosomal copy of a non-fluorescent EGFP (Y66S). In these cells, 293-iY66S, SpCas9 activity was measured by the recovery of EGFP fluorescence following the substitution of the mutated gene with a wild-type allele through SpCas9 mediated homology-directed repair in the presence of a co-transfected donor plasmid carrying a non-fluorescent fragment of wild-type EGFP. Compared to the conventional SpCas9 approach (sgCtr), the target specificity for EGFP homology-directed repair was improved by using the SLiCES circuitry (sgCas-a) by 4-fold (FIG. 2e and FIG. 6). Further improvement (7,5-fold) was obtained with the optimized version of sgCas-a (sgCas-a-opt) (FIG. 2e and FIG. 6), as previously observed in knock-out experiments.

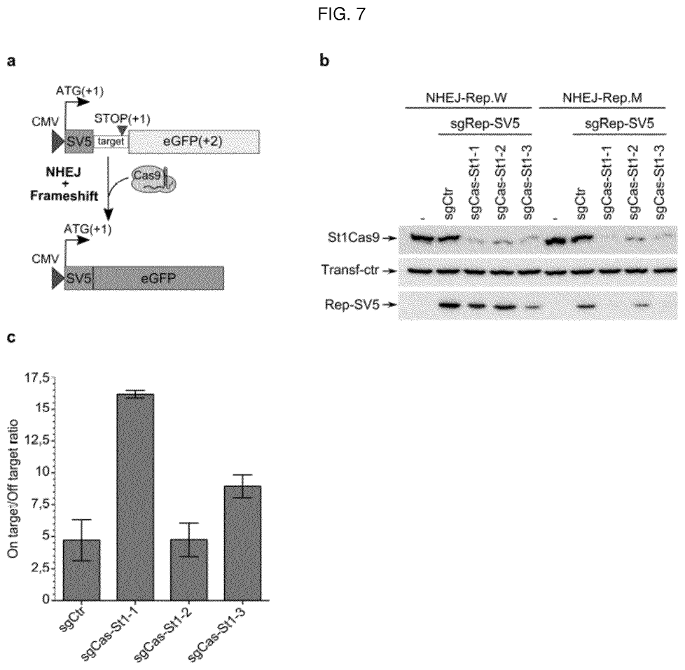

To demonstrate that the SLiCES methodology is readily transferrable to other RNA-guided nucleases, SLiCES was adapted to Cas9 from Streptococcus thermophilus (St1Cas9) by using specific sgRNAs (sgCas-St1-1, -2 and -3) to induce St1Cas9 downregulation (FIG. 7). Next, the target specificity of the conventional SpCas9 and the SLiCES circuit (sgCas-a) towards endogenous sequences was comparatively analyzed. Four genomic sites (VEGFA, ZSCAN and two targets in the EMX1 locus) and two previously validated off target sites (Kleinstiver, B. P. et al. Nature 2016, doi:10.1038/nature16526) for each sgRNA were analyzed by tracking indels by decomposition (TIDE) (Brinkman, E. K., et al., Nucleic Acids Res. 2014, 42, e168) revealing that the SLiCES approach improved cleavage specificity by approximately 1.5-2.5 fold (FIG. 2f).

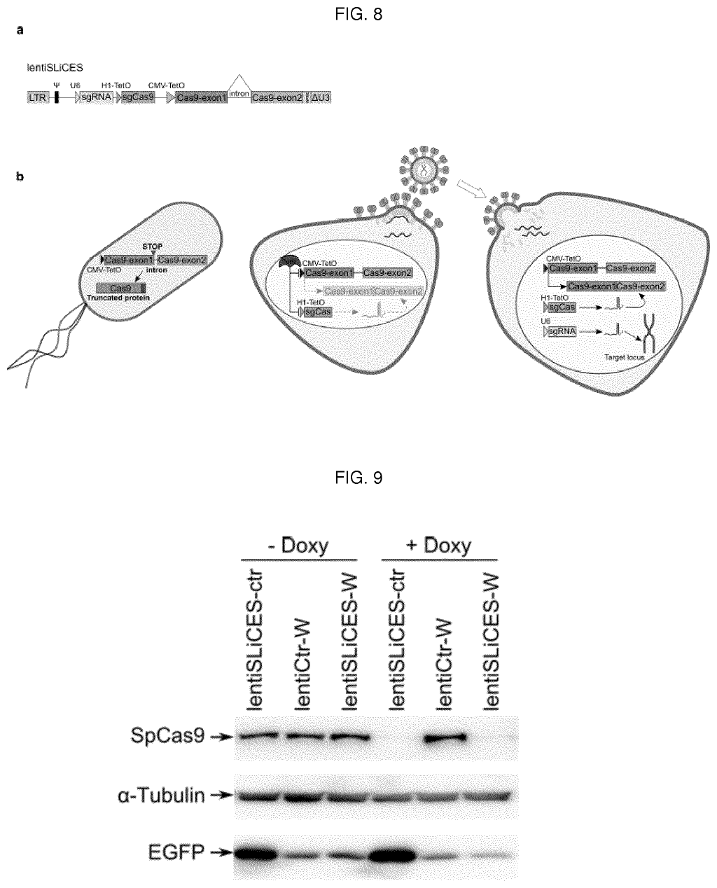

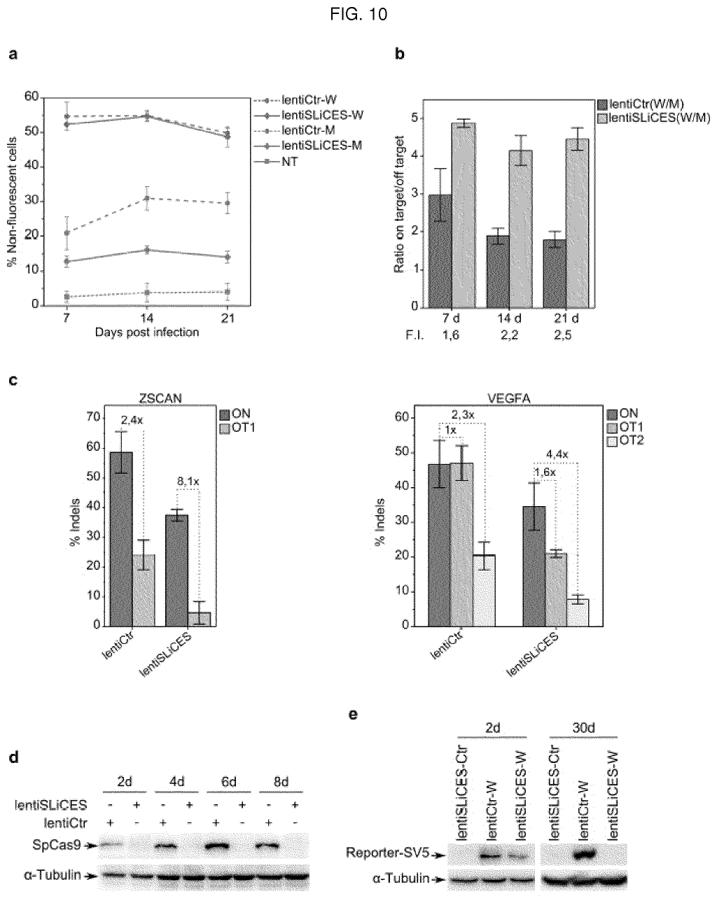

The self-limiting SpCas9/sgRNA circuitry with the best selected self-limiting sgRNA (sgCas-a-opt) was then transferred to a lentiviral system (FIG. 8) to generate lentiSLiCES. To avoid the leaky expression of SpCas9, and the consequent degradation of DNA during plasmid preparation in bacteria, an intron was introduced into the SpCas9 open reading frame to form an expression cassette divided in two exons (exon 1 and 2, schematized in FIG. 8). As splicing does not occur in bacteria, the transcripts produced are translated in bacteria as a catalytically inactive SpCas9 fragment. Next, to circumvent the self-cleavage activity during lentiviral vector production, Tetracycline inducible (TetO) promoters were introduced to regulate both SpCas9 and the self-targeting sgRNAs expression. The TetO promoter is negatively regulated by a specific repressor, TetR, which is expressed in producing cells and, in the absence of doxycycline, inhibits transcription through binding to tetracycline operator sequences located within the promoter region (schematized in FIG. 8b). The drop in SpCas9 intracellular levels in producing cells observed with the activation of the self-limiting circuitry with doxycycline demonstrates the strict requirement of the repressible promoters at viral production steps in order to obtain un-altered lentiSLiCES particles (FIG. 9). To evaluate the on/off target activity of the lentiSLiCES, the percentage of EGFP negative 293-multiEGFP cells was followed at different time points after transduction with self-limiting lentiviral vectors either carrying the specific sgRNA sgGFP-W (lentiSLiCES-W) or the mismatched sgGFP-M (lentiSLiCES-M) and compared with the effect obtained with non-self-limiting lentiviral vectors carrying the same sgRNAs towards EGFP (lentiCtr-W or -M). Both lentiCtr-W and lentiSLICES-W showed similarly stable on-target activity at all the time points within a 3 weeks period (FIG. 10a). Conversely, the percentage of EGFP cells unspecifically targeted by the sgGFP-M increased in time with the lentiCtr delivery system; this event was not observed with the same sgRNA delivered through lentiSLiCES throughout the 3 weeks period (FIG. 10a). Therefore, lentiSLiCES generated no off-target accumulation in time (compare day 7 and day 21, FIG. 10b). Consistently, at the end-point we observed the largest difference between the ratios of the EGFP negative cells obtained with the sgGFP-W over the sgGFP-M delivered either through the lentiSLICES (on/off ratio ˜5) or the lentiCtr systems (on/off ratio ˜2) (FIG. 10b). In agreement with these results the target specificity of the lentiSLiCES towards endogenous sequences (ZSCAN and VEGFA loci) showed significant improvement as compared to the non-self-limiting lentiCtr (approximately 2-4 fold) (FIG. 10c).

These data suggest that the decreased expression of SpCas9 obtained through the SLiCES circuit improves editing specificity. Indeed, at early time points (2 days post-transduction) SpCas9 protein was already much less present in cells treated with the lentiSLiCES than in cells treated with the non-self-limiting lentiviral control (lentiCtr) (FIG. 10d). Notably, in lentiCtr treated cells the levels of SpCas9 remained stable and higher than in lentiSLiCES treated cells where no nuclease could be detected at any later time point. To functionally assess the level of SpCas9 activity delivered through the lentiSLiCES, a non-homologous end joining (NHEJ) reporter plasmid (NHEJ-Rep.W) expressing the simian virus-5 tag fused with EGFP (SV5-EGFP) upon targeted nuclease activity (schematized in FIG. 7a) was employed. The NHEJ-Rep.W revealed that SpCas9 delivered through the lentiCtr was active at all time points following transduction, while the activity of SpCas9 carried by the lentiSLiCES was detected 2 days after transduction, but could not be observed at later time points (30 days) (FIG. 10e).

The limitations of in vivo SpCas9 applications clearly emerge from data of the present invention showing that long term nuclease expression delivered through lentiviral systems results in the accumulation of unwanted cleavages. This detrimental effect could not be overcome even with the recently developed, more specific SpCas9 variant, eSpCas9(1.1) (Slaymaker, I. M. et al. Science 2016, 351, 84-88). The self-limiting circuitry strategy, lentiSLiCES, of the present invention exploits the efficiency of viral based delivery and simultaneously limits the amount of SpCas9 post transduction and viral integration. By limiting in time and abundance Cas9 expression, SLiCES avoids the accumulation of off-target cleavages that instead are observed with the use of conventional Cas9 delivery approaches. To further improve the SLiCES strategy, Integrase Defective Lentiviral Vectors (IDLV) (Chick, H. E. et al. Hum. Gene Ther. 2012, 23, 1247-1257) could be used to maintain the viral-based efficiency in cellular delivery, while enhancing the transient peak-like nature of Cas9 expression. A variety of Cas9 applications, such as the regulation of gene expression obtained by the combination with transcriptional activation domains (Konermann, S. et al., Nature 2015, 517, 583-588; Mali, P. et al., Nat. Biotechnol. 2013, 31, 833-838; Hilton, I. B. et al., Nat. Biotechnol. 2015, 33, 510-517) might be significantly improved through their adaptation to lentiSLiCES. In fact, these approaches as well as the refined modulation of gene expression obtained with a genetic kill-switch circuit (Moore, R. et al., Nucleic Acids Res. 2015, 43, 1297-1303; Kiani, S. et al. Nat. Methods 2015, 12, 1051-1054) could be potentiated by a tunable self-limiting approach to restrict in time Cas9-mediated induction of the targeted cellular promoters. Finally, SLiCES may significantly improve some recently developed Cas9 genome engineering procedures that are susceptible to continuous nuclease activity. For instance, current techniques to efficiently substitute genomic sequences use Cas9 to increase the rate of homology-directed repair; nevertheless, these techniques are often limited by the continuous re-cleavage of the newly substituted genomic sequence by Cas9 (Paquet, D. et al., Nature 2016, 533, 125-129), which could be easily overcome by nuclease inactivation.

Supplemenary Discussion

Cas9 origin from prokaryotic cells, even after human codon optimization, allows to easily select several possible non-repetitive sgRNAs (as sgCas-a, -b, -c) with very few possible off-targets into the eukaryotic genome This implies that the possibility of generating potential new off-targets given the presence of a second sgRNA could be considered almost negligible.

As demonstrated by the improved performance obtained with St1Cas9 integrated within the self-limiting circuit, the SLiCES is proven to be easily adapted to the new emerging variants of nucleases (Esvelt, K. M. et al. Nat. Methods 2013, 10, 1116-1121; Zetsche, B. et al. Cell 2015, 163, 759-771; Ran, F. A. et al. Nature 2015, 520, 186-191; Kleinstiver, B. P. et al. Nature 2016, doi:10.1038/nature16526; Slaymaker, I. M. et al. Science 2016, 351, 84-88) and sgRNAs (Fu, Y., et al. Nat. Biotechnol. 2014, 32, 279-284) for safer genome editing.

The SLiCES system can be potentially applied also to others viral vectors used for delivering RNA guided nucleases, stepping up the specificity of genome editing through different delivery systems. An example are AAV vectors exploited for small Cas9 variants (such as SaCas9) (Friedland, A. E. et al. Genome Biol. 2015, 16, 257) for which an all-in-one AAV-SLiCES approach is conceivable simply by transferring the technologies developed for lentiSLiCES. Taking in account the high propensity of AAV vectors to transduce cells with high multiplicity of infection (Ruozi, G. et al. Nat. Commun. 2015, 6, 7388), it is possible to design a delivery strategy for the SLiCES system for large size nucleases, such as SpCas9, StCas9 or AsCpf1, based on a mixture of two AAVs: one for delivering the nuclease only and a second vector carrying the self-limiting and the targeting gRNAs. This approach would be similar to the multiple plasmid system presented in FIG. 2.

Methods

Plasmids and Oligonucleotides.

The 3XFLAG-tagged S. pyogenes Cas9 was expressed from the pX-Cas9 plasmid, which was obtained by removal of an Ndel fragment including the sgRNA expression cassette from pX330 (a gift from Feng Zhang, Addgene #42230) (Cong, L. et al., Science 2013, 339, 819-823). The sgRNAs were transcribed from a U6 promoter driven cassette, derived from px330 and cloned into pUC19. sgRNA oligos were cloned using a double Bbsl site inserted before the sgRNA constant portion by a previously published cloning strategy (Cong, L. et al., Science 2013, 339, 819-823). Plasmids expressing FLAG-tagged S. thermophilus Cas9 (pJDS246-CMV-St1-Cas9) and S. thermophilus gRNA (pMLM3636-U6-+103gRNA_St1Cas9) were a gift of Claudio Mussolino (Müller, M. et al. Mol. Ther. J. Am. Soc. Gene Ther. 2016, 24, 636-644). S. thermophilus sgRNAs oligos were cloned into pMLM3636-U6-+103gRNA_St1Cas9 using BsmBI and transcribed from a U6 promoter. The list of sgRNAs and target sites employed in this study is available in Table 1.

| SpCas9 | name | protospacer (*) | target (**) |
|---|---|---|---|
| | GFPW | gCTCGTGACCACCCTGACCTA | accCTCGTGACCACCCTGACCTACGGcgt |
| | | (SEQ ID N. 11) | (SEQ ID N. 12) |
| | Rep. SV5 | | agcCTCGTGACCACCCTGACCTACGGagt |
| | | | (SEQ ID N. 13) |
| | GFPM | 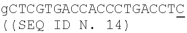 | |
| | GFPMM | 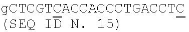 | |
| | VEGFA | GGTGAGTGAGTGTGTGCGTG | gtgGGTGAGTGAGTGTGTGCGTGTGGggt |
| | | (SEQ ID N. 16) | (SEQ ID N. 17) |
| | VEGFA OT1 | | 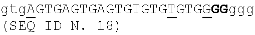 |
| | VEGFA OT2 | | 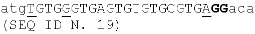 |
| | ZSCAN | GTGCGGCAAGAGCTTCAGCC | catGTGCGGCAAGAGCTTCAGCCGGGgct |
| | | (SEQ ID N. 20) | (SEQ ID NO. 21) |
| | ZSCAN OT1 | | 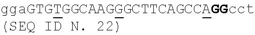 |
| | ZSCAN OT2 | | 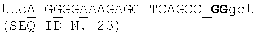 |
| | EMX1-k | GAGTCCGAGCAGAAGAAGAA | cctGAGTCCGAGCAGAAGAAGAAGGGctc |
| | | (SEQ ID N. 24) | (SEQ ID N. 25) |
| | EMX1-k OT2 | | 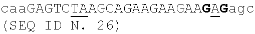 |
| | EMX1-k OT1 | | 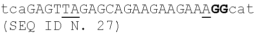 |
| | EMX1-r | GGCCTCCCCAAAGCCTGGCCA | cagGCCTCCCCAAAGCCTGGCCAGGGagt |
| | | (SEQ ID N. 28) | (SEQ ID N. 29) |
| | EMX1-r OT1 | | 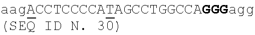 |
| | EMX1-r OT2 | | 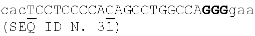 |
| | Cas-a | gTACGCCGGCTACATTGACGG | ggcTACGCCGGCTACATTGACGGcgg |
| | | (SEQ ID 1) | (SEQ ID N. 32) |
| | Cas-b | GATCCTTGTAGTCTCCGTCG | catGATCCTTGTAGTCTCCGTCGTGGtcc |
| | | (SEQ ID 2) | (SEQ ID N. 33) |
| | Cas-c | GGCTACGCCGGCTACATTGA | aacGGCTACGCCGGCTACATTGACGGcgg |
| | | (SEQ ID 3) | (SEQ ID N. 34) |
| | control | GGGTCTTCGAGAAGACCT | |
| | | (SEQ ID N. 35) | |
| STh1Cas9 | | | |
| | NHEJ-Rep. W | GTCCCCTCCACCCCACAGTG | agaGTCCCCTCCACCCCACAGTGCAAGAAAtcc |
| | | (SEQ ID N. 36) | (SEQ ID N. 37) |
| | NHEJ-Rep. M | | 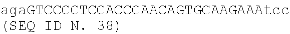 |
| | STh1-1 | GGCAGAAGGCTGACCCGGCG | cagGGCAGAAGGCTGACCCGGCGGAAGAAAcac |
| | | (SEQ ID 4) | (SEQ ID N. 39) |
| | STh1-2 | gGCCTACAGAAGCGAGGCCC | agcGCCTACAGAAGCGAGGCCCTGAGAATcct |
| | | (SEQ ID 5) | (SEQ ID N. 40) |
| | STh1-3 | gAGACTAACGAGGACGACGA | cgcGAGACTAACGAGGACGACGAGAAGAAAgcc |
| | | (SEQ ID 6) | (SEQ ID N. 41) |
| | control | GAGACGATTAATGCGTCTC | |
| | | (SEQ ID N. 42) | |

(*) Lowercase indicates non-matching additional 5′g.

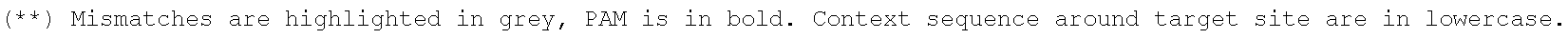

pcDNA5-FRT-TO-EGFP plasmid was obtained by subcloning EGFP from pEGFP-N1 in a previously published vector (Vecchi, L., et al. J. Biol. Chem. 2012, 287, 20007-20015) derived from pcDNA5-FRT-TO (Invitrogen). pcDNA5-FRT-TO-EGFP-Y66S was obtained by site directed mutagenesis of pcDNA5-FRT-TO-EGFP. A sgRNA resistant, non-fluorescent truncated EGFP fragment (1-T203K-stop), obtained by site directed mutagenesis of the pcDNA5-FRT-TO-EGFP plasmid, was amplified by PCR and inserted in place of EGFP in the pcDNA5-FRT-TO-EGFP plasmid, yielding the donor pcDNA5-FRT-TO-rEGFP(1-T203K-stop) plasmid. The SV5-EGFP-based NHEJ reporters employed in this application (Rep. SV5, NHEJ-REP.W and NHEJ-Rep.M) were generated by cloning into the Nhel/BspEl sites dsDNA oligos corresponding to the complete target sequence (including PAM) recognized by a sgRNA of interest. The target is inserted between the SV5 tag and EGFP coding sequences, with the EGFP sequence positioned out of frame with respect to the starting ATG codon of the SV5 tag open reading frame (ORF). A stop codon is inserted in the SV5 frame, immediately after the target sequence. The pcDNA3 MHC-I-roTag plasmid is described in Petris, G., et al., PloS One 2014, 9, e96700. Information on plasmids DNA sequences produced for experiments described in this application are found in Supplementary Sequences and Sequence Listing.

Cell Culture and Transfections. 293T/17 cells were obtained from ATCC. 293TR cells, constitutively expressing the Tet repressor (TetR), were generated by lentiviral transduction of parental 293T/17 cells using the pLenti-CMV-TetR-Blast vector (a gift from Eric Campeau, Addgene #17492) (Campeau, E. et al. P/oS One 2009, 4, e6529) and were pool selected with 5 μg/ml of blasticidin (Life Technologies). 293-multiEGFP cells were generated by stable transfection of pEGFP-IRES-Puromicin and selected with 1 μg/ml of puromicin. 293-iEGFP and 293-iY66S cells (Flp-In T-REx system; Life Technologies) were generated by Flp-mediated recombination using the pcDNA5-FRT-TO-EGFP or the pcDNA5-FRT-TO-EGFP-Y66S as donor plasmids, respectively, in cells carrying a single genomic FRT site and stably expressing the tetracycline repressor (293 T-Rex Flp-In, cultured in selective medium containing 15 μg/ml blasticidin and 100 μg/ml zeocin-Life Technologies). 293-iEGFP and 293-iY66S were cultured in selective medium containing 15 μg/ml blasticidin and 100 μg/ml hygromycin (Life Technologies). 293-iEGFP and 293-iY66S selected clones were checked for integration specificity by loss of zeocin resistance. All cell lines were cultured in DMEM supplemented with 10% FBS, 2 mM L-Gln, 10 U/ml penicillin, and 10 μg/ml streptomycin and the appropriate antibiotics indicated above. 293T, 293-iEGFP or 293-iY66S cells were transfected in 12 or 24 multi wells with 250-500 ng of pX-Cas9 and 250-500 ng of the desired pUC19-sgRNA plasmid using TranslT-LT1 (Mirus Bio), according to manufacturer's instructions. Cells were collected 2-4 days after transfection or as indicated.

In 293-iEGFP and 293-iY66S cells the expression of EGFP was induced by treatment with 100 ng/ml doxycycline (Cayman Chemical) for 20 h before fluorescence measurement.

lentiSLiCES Vectors.

lentiSLiCES was prepared from lentiCRISPRv1 transfer vector by substituting the EFS-SpCas9-2A-Puro cassette with a SpCas9(intron)-IRES-Blasticidin fragment together with a CMV-TetO promoter. The intron introduced in SpCas9 (see Supplementary sequence information) derives from the mouse immunoglobulin heavy chain precursor V-region intron (GenBank ID: M12880.1), previously used with different flanking exons (Vecchi, L., et al., J. Biol. Chem. 2012, 287, 20007-20015; Petris, G., et al., PloS One 2014, 9, e96700; Li, E. et al. Protein Eng. 1997, 10, 731-736). The EMCV-IRES regulating the translation of a blasticidin resistance gene was cloned downstream of SpCas9 to allow the antibiotic selection of transduced cells, even after the generation of frameshift mutations following Cas9 self-cleavage of the integrated vector.

The sgCtr-opt or the sgCas9-a-opt were assembled with an H1-TetO promoter within the pUC19 plasmid, PCR amplified and then cloned into a unique EcoRl site in lentiCRISPRv1 and selected for the desired orientation. The sgRNAs targeting the chosen locus were cloned into the lentiCRISPRv1 sgRNA cassette using the two BsmBI sites, following standard procedures (Brinkman, E. K., et al., Nucleic Acids Res. 2014, 42, e168).

Information on DNA sequences of lentiSLiCES can be found in Supplementary Supplementary Sequences and Sequence Listing.

Lentiviral Vector Production.

Lentiviral particles were produced by seeding 4×10⁶ 293 T or 293TR cells into a 10 cm dish, for lentiCRISPR or lentiSLiCES production, respectively. The day after the plates were transfected with 10 μg of each transfer vector together with 6.5 μg pCMV-deltaR8.91 packaging vector and 3.5 μg pMD2.G using the polyethylenimine (PEI) method (Casini, A., etal., J. Virol. 2015, 89, 2966-2971). After an overnight incubation, the medium was replaced with fresh complete DMEM and 48 hours later the supernatant containing the viral particles was collected, spun down at 500×g for 5 minutes and filtered through a 0.45 μm PES filter.

After collection, lentiSLiCES viral vectors were concentrated using polyethylene glycol (PEG) 6000 (Sigma). Briefly, a 40% PEG 6000 solution in water was mixed in a 1:3 ratio with the vector-containing supernatant and incubated for 3 hours to overnight at 4° C. Subsequently, the mix was spun down for 45 minutes at 2000×g in a refrigerated centrifuge. The pellets were then resuspended in a suitable volume of DMEM complete medium. lentiCRISPR vectors were used unconcentrated. The titer of the lentiviral vectors (reverse transcriptase units, RTU) was measured using the product enhanced reverse transcriptase (PERT) assay (Francis, A. C. et al. AIDS Res. Hum. Retroviruses 2014, 30, 717-726).

Infections and EGFP Fluorescence Detection.

One day before transduction 10⁵ 293 T, 293-iEGFP or 293-multiEGFP cells were seeded in a 24-well plate. For lentiSLiCES vectors, cells were transduced by centrifuging 2 RTU/well for 2 hours at 1600×g at 16° C., and then leaving the vectors incubating with the cultures for an overnight. Starting from 24 hours post transduction onwards the cultures were selected with 5 μg/ml of blasticidin, where needed. For lentiCRISPR vectors, 0.5 RTU/well were used following the same transduction protocol and cells were selected with 0.5 μg/ml of puromycin.

When targeting genomic EGFP sequences, cells were collected and analyzed using a FACSCanto flow cytometer (BD Biosciences) to quantify the percentage of EGFP loss or induction (gene substitution experiments).

Western Blots.

Cells were lysed in NEHN buffer (20 mM HEPES pH 7.5, 300 mM NaCl, 0.5% NP40, NaCl, 1 mM EDTA, 20% glycerol supplemented with 1% of protease inhibitor cocktail (Pierce)). Cell extracts were separated by SDS-PAGE using the PageRuler Plus Protein Standards as the standard molecular mass markers (Thermo Fisher Scientific). After electrophoresis, samples were transferred to 0.22 μm PVDF membranes (GE Healthcare). The membranes were incubated with mouse anti-FLAG (Sigma) for detecting SpCas9 and St1Cas9, mouse anti-α-tubulin (Sigma), rabbit anti-GFP (Santa Cruz Biotechnology), mouse anti-roTag mAb (Petris, G., et al., PloS One 2014, 9, e96700) and with the appropriate HRP conjugated goat anti-mouse (KPL) or goat anti-rabbit (Santa Cruz Biotechnology) secondary antibodies for ECL detection. Images were acquired and bands were quantified using the UVltec Alliance detection system.

Detection of Cas9-Induced Genomic Mutations.

Genomic DNA was isolated at 72 h post-transfection or as indicated for transduction experiments, using the DNeasy® Blood & Tissue kit (Qiagen). PCR reactions to amplify genomic loci were performed using the Phusion High-Fidelity DNA polymerase (Thermo Fisher). Samples were amplified using the oligos listed in Table 2. Purified PCR products were analyzed either by sequencing and applying the TIDE tool (Chen, B., etal. Cell 2013, 155, 1479-1491) or by T7 Endonuclease 1 (T7E1) assay (New England BioLabs). In the latter case PCR amplicons were denatured and re-hybridized before digestion with T7E1 for 30 min at 37° C. Digested material was separated using standard agarose gel and quantified using the ImageJ software. Indel formation was calculated according to the following equation: % gene modification=100×(1−(1−fraction cleaved)^{1/2}).

TABLE 2
Sequences of the oligos used to amplify EGFP, the genomic loci (VEGF-
A, ZSCAN, EMX) and relative off target sites.

| locus | oligo1 | oligo2 |
|---|---|---|
| GFP | ACCATGGTGAGCAAGGGCGAGGA | AGCTCGTCCATGCCGAGAGTGATC |
| | (SEQ ID N. 43) | (SEQ ID N. 44) |
| VEGFA | GCATACGTGGGCTCCAACAGGT | CCGCAATGAAGGGGAAGCTCGA |
| | (SEQ ID N. 45) | (SEQ ID N. 46) |
| VEGFA OT1 | CAGGCGCCTTGGGCTCCGTCA | CCCCAGGATCCGCGGGTCAC |
| | (SEQ ID N. 47) | (SEQ ID N. 48) |
| VEGFA OT2 | AGTCAGCCCTCTGTATCCCTGGA | GAGATATCTGCACCCTCATGTTCAC |
| | (SEQ ID N. 49) | (SEQ ID N. 50) |
| ZSCAN | GACTGTGGGCAGAGGTTCAGC | TGTATACGGGACTTGACTCAGACC |
| | (SEQ ID N. 51) | (SEQ ID N. 52) |
| ZSCAN OT1 | CACGACTGCAGGCTCATGAGC | GAAGCGCTTACCACACACATCAC |
| | (SEQ ID N. 53) | (SEQ ID N. 54) |
| ZSCAN OT2 | AGTCACATGCTGCCTGGATTGAC | GTGGAGGAGATTTCTCTAGGAGAG |
| | (SEQ ID N. 55) | (SEQ ID N. 56) |
| EMX1 | CTGCCATCCCCTTCTGTGAATGT | GGAATCTACCACCCCAGGCTCT |
| | (SEQ ID N. 57) | (SEQ ID N. 58) |
| EMX1-k OT2 | CTGCTGTTTCCTGAAGCTGCCACT | CTGCCATGGAAATTCCAGAGGGAAC |
| | (SEQ ID N. 59) | (SEQ ID N. 60) |
| EMX1-k OT1 | TGTGGGGAGATTTGCATCTGTGGA | TTGAGACATGGGGATAGAATCATGAAC |
| | (SEQ ID N. 61) | (SEQ ID N. 62) |
| EMX1-r OT1 | TGAACGAATCAGGTCTGAGAGGATC | GAGCTTCACTCCAGAGAGGCTGT |
| | (SEQ ID N. 63) | (SEQ ID N. 64) |
| EMX1-r OT2 | TGCTACTGCTGGCTGCAGAGATG | GCATTCGTTTTGGGAGGCAGAGGA |
| | (SEQ ID N. 65) | (SEQ ID N. 66) |

Supplementary sequences

A subset of new plasmids produced for this manuscript:
Rep. SV5-EGFP (SEQ ID N.67)
Donor pcDNA5-FRT-TO-rEGFP(1-T203K-stop) (SEQ ID N.68)
LentiSLiCES (SEQ ID N.10)

Rep. SV5-EGFP (Nhel and BspEl restriction sites to clone target sequence are underlined, in frame stop condon is in bold).

SV5, [symbol] , linker, [symbol] [this EGFP CDS, resistant to specific sgRNAs targeting EGFP (sgGFP-W, -M) for which the target sequences were initially cloned into the reporter target region, was obtained by introducing the synonymous mutations that are indicated in lowercase bold to prevent its targeting]

SEQ ID N. 67:

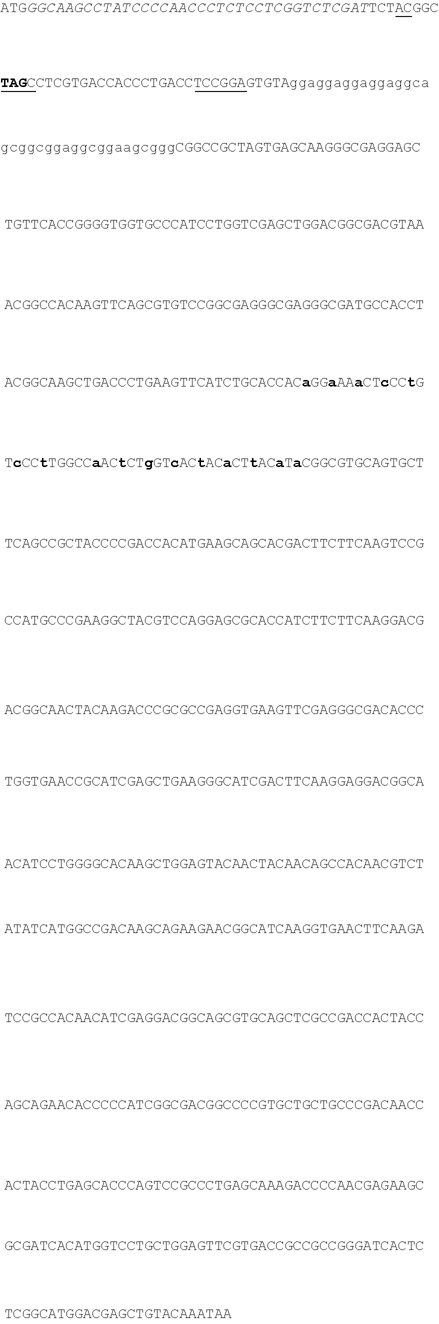

Donor pcDNA5-FRT-TO-rEGFP(1-T203K-stop) plasmid

rEGFP(1-T203K-stop) donor, synonymous codons employed to prevent sgRNA retargeting after homologous recombination are highlighted in lowercase bold, the key nucleotide change to restore EGFP fluorescence by reverting the Y66S mutation is underlined. The end of then 410 bp 3′-homology arm (corresponding to T203K-stop) is highlighted in [symbol] .

ATGGTGAGCAAGGGCGAGGAGCTGTTCACCGGGGTGGTGCCCATCCT
GGTCGAGCTGGACGGCGACGTAAACGGCCACAAGTTCAGCGTGTCCG
GCGAGGGCGAGGGCGATGCCACCTACGGCAAGCTGACCCTGAAGTTC
ATCTGCACCACaGGaAAaCTcCCtGTcCCtTGGCCaACtCTgGTcAC
tACaCTtACaTaCGGCGTGCAGTGCTTCAGCCGCTACCCCGACCACA
TGAAGCAGCACGACTTCTTCAAGTCCGCCATGCCCGAAGGCTACGTC
CAGGAGCGCACCATCTTCTTCAAGGACGACGGCAACTACAAGACCCG
CGCCGAGGTGAAGTTCGAGGGCGACACCCTGGTGAACCGCATCGAGC
TGAAGGGCATCGACTTCAAGGAGGACGGCAACATCCTGGGGCACAAG
CTGGAGTACAACTACAACAGCCACAACGTCTATATCATGGCCGACAA
GCAGAAGAACGGCATCAAGGTGAACTTCAAGATCCGCCACAACATCG
AGGACGGCAGCGTGCAGCTCGCCGACCACTACCAGCAGAACACCCCC

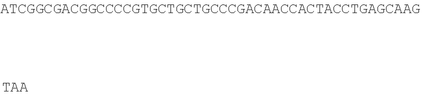

LentiSLiCES

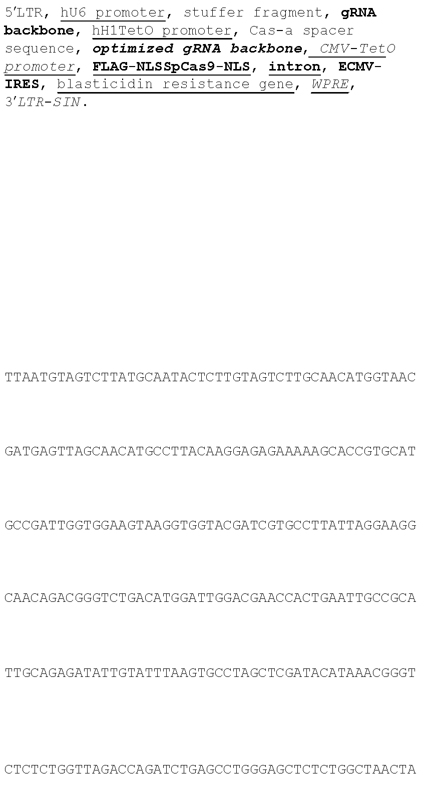

GGGAACCCACTGCTTAAGCCTCAATAAAGCTTGCCTTGAGTGCTTCA
AGTAGTGTGTGCCCGTCTGTTGTGTGACTCTGGTAACTAGAGATCCC
TCAGACCCTTTTAGTCAGTGTGGAAAATCTCTAGCAGTGGCGCCCGA
ACAGGGACTTGAAAGCGAAAGGGAAACCAGAGGAGCTCTCTCGACGC
AGGACTCGGCTTGCTGAAGCGCGCACGGCAAGAGGCGAGGGGCGGCG
ACTGGTGAGTACGCCAAAAATTTTGACTAGCGGAGGCTAGAAGGAGA
GAGATGGGTGCGAGAGCGTCAGTATTAAGCGGGGGAGAATTAGATCG
CGATGGGAAAAAATTCGGTTAAGGCCAGGGGGAAAGAAAAAATATAA
ATTAAAACATATAGTATGGGCAAGCAGGGAGCTAGAACGATTCGCAG
TTAATCCTGGCCTGTTAGAAACATCAGAAGGCTGTAGACAAATACTG
GGACAGCTACAACCATCCCTTCAGACAGGATCAGAAGAACTTAGATC
ATTATATAATACAGTAGCAACCCTCTATTGTGTGCATCAAAGGATAG
AGATAAAAGACACCAAGGAAGCTTTAGACAAGATAGAGGAAGAGCAA
AACAAAAGTAAGACCACCGCACAGCAAGCGGCCGCTGATCTTCAGAC
CTGGAGGAGGAGATATGAGGGACAATTGGAGAAGTGAATTATATAAA
TATAAAGTAGTAAAAATTGAACCATTAGGAGTAGCACCCACCAAGGC
AAAGAGAAGAGTGGTGCAGAGAGAAAAAAGAGCAGTGGGAATAGGAG
CTTTGTTCCTTGGGTTCTTGGGAGCAGCAGGAAGCACTATGGGCGCA
GCGTCAATGACGCTGACGGTACAGGCCAGACAATTATTGTCTGGTAT
AGTGCAGCAGCAGAACAATTTGCTGAGGGCTATTGAGGCGCAACAGC
ATCTGTTGCAACTCACAGTCTGGGGCATCAAGCAGCTCCAGGCAAGA
ATCCTGGCTGTGGAAAGATACCTAAAGGATCAACAGCTCCTGGGGAT
TTGGGGTTGCTCTGGAAAACTCATTTGCACCACTGCTGTGCCTTGGA
ATGCTAGTTGGAGTAATAAATCTCTGGAACAGATTTGGAATCACACG
ACCTGGATGGAGTGGGACAGAGAAATTAACAATTACACAAGCTTAAT
ACACTCCTTAATTGAAGAATCGCAAAACCAGCAAGAAAAGAATGAAC
AAGAATTATTGGAATTAGATAAATGGGCAAGTTTGTGGAATTGGTTT
AACATAACAAATTGGCTGTGGTATATAAAATTATTCATAATGATAGT
AGGAGGCTTGGTAGGTTTAAGAATAGTTTTTGCTGTACTTTCTATAG
TGAATAGAGTTAGGCAGGGATATTCACCATTATCGTTTCAGACCCAC
CTCCCAACCCCGAGGGGACCCAGGAGGCCTATTTCCCATGATTCCTT
CATATTTGCATATACGATACAAGGCTGTTAGAGAGATAATTAGAATT
AATTTGACTGTAAACACAAAGATATTAGTACAAAATACGTGACGTAG
AAAGTAATAATTTCTTGGGTAGTTTGCAGTTTTAAAATTATGTTTTA
AAATGGACTATCATATGCTTACCGTAACTTGAAAGTATTTCGATTTC

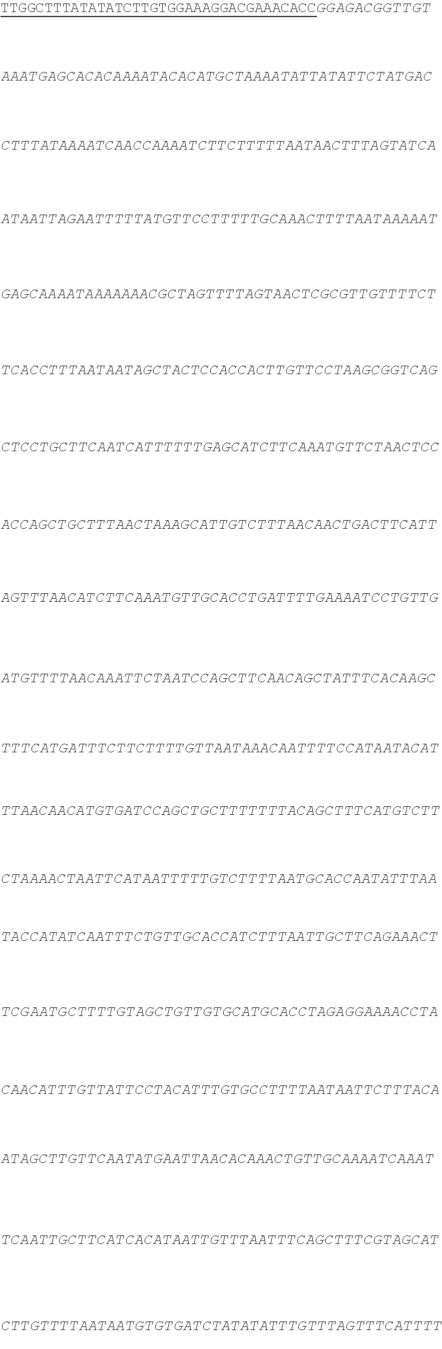

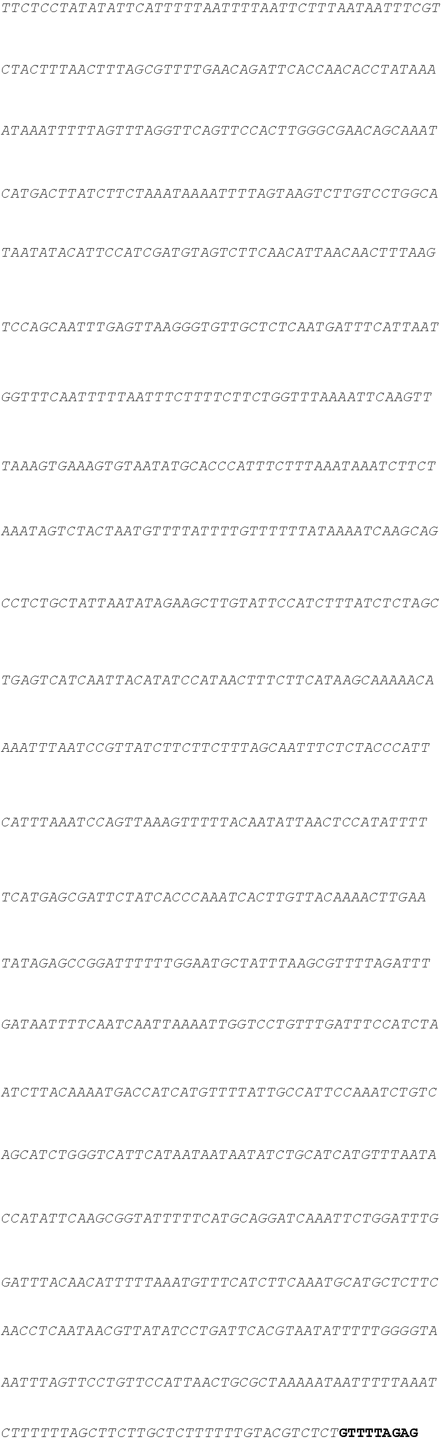

CTAGAAATAGCAAGTTAAAATAAGGCTAGTCCGTTATCAACTTGAAA
AAAGTGGCACCGAGTCGGTGCTTTTTTGaattctagtagaattgagg

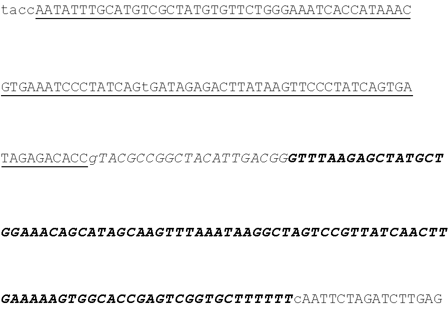

ACAAATGGCAGTATTCATCCACAATTTTAAAAGAAAAGGGGGGATTG
GGGGGTACAGTGCAGGGGAAAGAATAGTAGACATAATAGCAACAGAC
ATACAAACTAAAGAATTACAAAAACAAATTACAAAAATTCAAAATTT
TCGGGTTTATTACAGGGACAGCAGAGATCCACTTTGGCGCCGGCtcg
agGTTGACATTGATTATTGACTAGTTATTAATAGTAATCAATTACGG
GGTCATTAGTTCATAGCCCATATATGGAGTTCCGCGTTACATAACTT
ACGGTAAATGGCCCGCCTGGCTGACCGCCCAACGACCCCCGCCCATT
GACGTCAATAATGACGTATGTTCCCATAGTAACGCCAATAGGGACTT
TCCATTGACGTCAATGGGTGGAGTATTTACGGTAAACTGCCCACTTG
GCAGTACATCAAGTGTATCATATGCCAAGTACGCCCCCTATTGACGT
CAATGACGGTAAATGGCCCGCCTGGCATTATGCCCAGTACATGACCT
TATGGGACTTTCCTACTTGGCAGTACATCTACGTATTAGTCATCGCT
ATTACCATGGTGATGCGGTTTTGGCAGTACATCAATGGGCGTGGATA
GCGGTTTGACTCACGGGGATTTCCAAGTCTCCACCCCATTGACGTCA
ATGGGAGTTTGTTTTGGCACCAAAATCAACGGGACTTTCCAAAATGT
CGTAACAACTCCGCCCCATTGACGCAAATGGGCGGTAGGCGTGTACG
GTGGGAGGTCTATATAAGCAGAGCTCTCCCTATCAGTGATAGAGATC
TCCCTATCAGTGATAGAGATCGTCGACGAGCTCGTTTAGTGAACCGT
CAGATCGCCTGGAGAggatcCGCCACCATGGATTACAAAGACGATGA
CGATAAGATGGCCCCAAAGAAGAAGCGGAAGGTCGGTCGGTATCCAC
GGAGTCCCAGCAGCCGACAAGAAGTACAGCATCGGCCTGGACATCGG
CACCAACTCTGTGGGCTGGGCCGTGATCACCGACGAGTACAAGGTGC
CCAGCAAGAAATTCAAGGTGCTGGGCAACACCGACCGGCACAGCATC
AAGAAGAACCTGATCGGAGCCCTGCTGTTCGACAGCGGCGAAACAGC
CGAGGCCACCCGGCTGAAGAGAACCGCCAGAAGAAGATACACCAGAC
GGAAGAACCGGATCTGCTATCTGCAAGAGATCTTCAGCAACGAGATG
GCCAAGGTGGACGACAGCTTCTTCCACAGACTGGAAGAGTCCTTCCT
GGTGGAAGAGGATAAGAAGCACGAGCGGCACCCCATCTTCGGCAACA
TCGTGGACGAGGTGGCCTACCACGAGAAGTACCCCACCATCTACCTG
AGAAAGAAACTGGTGGACAGCACCGACAAGGCCGACCTGCGGCTGAT
CTATCTGGCCCTGGCCCACATGATCAAGTTCCGGGGCCACTTCCTGA
TCGAGGGCGACCTGAACCCCGACAACAGCGACGTGGACAAGCTGTTC
ATCCAGCTGGTGCAGACCTACAACCAGCTGTTGAGGAAAACCCCATC
AACGCCAGCGGCGTGGACGCCAAGCCATCCTGTCTGCCAGACTGAGC
AAGAGCAGACGGCTGGAAAATCTGATCGCCCAGCTGCCCGGCGAGAA
GAAGAATGGCCTGTTCGGCAACCTGATTGCCCTGAGCCTGGGCCTGA
CCCCCAACTTCAAGAGCAACTTCGACCTGGCGAGGATGCCAAACTGC
AGCTGAGCAAGGACACCTACGACGACGACCTGGACAACCTGCTGGCC
CAGATCGGCGACCAGTACGCCGACCTGTTTCTGGCCGCCAAGAACCT
GTCCGACGCCATCCTGCTGAGCGACATCCTGAGAGTGAACACCGAGA
TCACCAAGGCCCCCCTGAGCGCCTCTATGATCAAGAGATACGACGAG
CACCACCAGGACCTGACCCTGCTGAAAGCTCTCGTGCGGCAGCAGCT
GCCTGAGAAGTACAAAGAGATTTTCTTCGACCAGAGCAAGAACGGCT
ACGCCGGCTACATTGACGGCGGAGCCAGCCAGGAAGAGTTCTACAAG
TTCATCAAGCCCATCCTGGAAAAGATGGACGGCACCGAGGAACTGCT
CGTGAAGCTGAACAGAGAGGACCTGCTGCGGAAGCAGCGGACCTTCG
ACAACGGCAGCATCCCCCACCAGATCCACCTGGGAGAGCTGCACGCC
ATTCTGCGGCGGCAGGAAGATTTTTACCCATTCCTGAAGGACAACCG
GGAAAAGATCGAGAAGATCCTGACCTTCCGCATCCCCTACTACGTGG
GCCCTCTGGCCAGGGGAAACAGCAGATTCGCCTGGATGACCAGAAAG
AGCGAGGAAACCATCACCCCCTGGAACTTCGAGGAAGTGGTGGACAA
GGGCGCTTCCGCCCAGAGCTTCATCGAGCGGATGACCAACTTCGATA
AGAACCTGCCCAACGAGAAGGTGCTGCCCAAGCACAGCCTGCTGTAC
GAGTACTTCACCGTGTATAACGAGCTGACCAAAGTGAAATACGTGAC
CGAGGGAATGAGAAAGCCCGCCTTCCTGAGCGGCGAGCAGAAAAAGG
CCATCGTGGACCTGCTGTTCAAGACCAACCGGAAAGTGACCGTGAAG
CAGCTGAAAGAGGACTACTTCAAGAAAATCGAGTGCTTCGACTCCGT
GGAAATCTCCGGCGTGGAAGATCGGTTCAACGCCTCCCTGGGCACAT
ACCACGATCTGCTGAAAATTATCAAGGACAAGGACTTCCTGGACAAT
GAGGAAAACGAGGACATTCTGGAAGATATCGTGCTGACCCTGACACT
GTTTGAGGACAGAGAGATGATCGAGGAACGGCTGAAAACCTATGCCC
ACCTGTTCGACGACAAAGTGATGAAGCAGCTGAAGCGGCGGAGATAC
ACCGGCTGGGGCAGGCTGAGCCGGAAGCTGATCAACGGCATCCGGGA
CAAGCAGTCCGGCAAGACAATCCTGGATTTCCTGAAGTCCGACGGCT
TCGCCAACAGAAACTTCATGCAGCTGATCCACGACGACAGCCTGACC

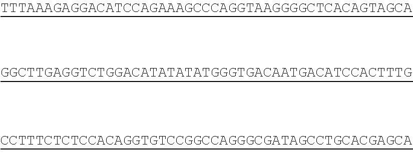

CATTGCCAATCTGGCCGGCAGCCCCGCCATTAAGAAGGGCATCCTGC
AGACAGTGAAGGTGGTGGACGAGCTCGTGAAAGTGATGGGCCGGCAC
AAGCCCGAGAACATCGTGATCGAAATGGCCAGAGAGAACCAGACCAC
CCAGAAGGGACAGAAGAACAGCCGCGAGAGAATGAAGCGGATCGAAG
AGGGCATCAAAGAGCTGGGCAGCCAGATCCTGAAAGAACACCCCGTG
GAAAACACCCAGCTGCAGAACGAGAAGCTGTACCTGTACTACCTGCA
GAATGGGCGGGATATGTACGTGGACCAGGAACTGGACATCAACCGGC
TGTCCGACTACGATGTGGACCATATCGTGCCTCAGAGCTTTCTGAAG
GACGACTCCATCGACAACAAGGTGCTGACCAGAAGCGACAAGAACCG
GGGCAAGAGCGACAACGTGCCCTCCGAAGAGGTCGTGAAGAAGATGA
AGAACTACTGGCGGCAGCTGCTGAACGCCAAGCTGATTACCCAGAGA
AAGTTCGACAATCTGACCAAGGCCGAGAGAGGCGGCCTGAGCGAACT
GGATAAGGCCGGCTTCATCAAGAGACAGCTGGTGGAAACCCGGCAGA
TCACAAAGCACGTGGCACAGATCCTGGACTCCCGGATGAACACTAAG
TACGACGAGAATGACAAGCTGATCCGGGAAGTGAAAGTGATCACCCT
GAAGTCCAAGCTGGTGTCCGATTTCCGGAAGGATTTCCAGTTTTACA
AAGTGCGCGAGATCAACAACTACCACCACGCCCACGACGCCTACCTG
AACGCCGTCGTGGGAACCGCCCTGATCAAAAAGTACCCTAAGCTGGA
AAGCGAGTTCGTGTACGGCGACTACAAGGTGTACGACGTGCGGAAGA
TGATCGCCAAGAGCGAGCAGGAAATCGGCAAGGCTACCGCCAAGTAC
TTCTTCTACAGCAACATCATGAACTTTTTCAAGACCGAGATTACCCT
GGCCAACGGCGAGATCCGGAAGCGGCCTCTGATCGAGACAAACGGCG
AAACCGGGGAGATCGTGTGGGATAAGGGCCGGGATTTTGCCACCGTG
CGGAAAGTGCTGAGCATGCCCCAAGTGAATATCGTGAAAAAGACCGA
GGTGCAGACAGGCGGCTTCAGCAAAGAGTCTATCCTGCCCAAGAGGA
ACAGCGATAAGCTGATCGCCAGAAAGAAGGACTGGGACCCTAAGAAG
TACGGCGGCTTCGACAGCCCCACCGTGGCCTATTCTGTGCTGGTGGT
GGCCAAAGTGGAAAAGGGCAAGTCCAAGAAACTGAAGAGTGTGAAAG
AGCTGCTGGGGATCACCATCATGGAAAGAAGCAGCTTCGAGAAGAAT
CCCATCGACTTTCTGGAAGCCAAGGGCTACAAAGAAGTGAAAAAGGA
CCTGATCATCAAGCTGCCTAAGTACTCCCTGTTCGAGCTGGAAAACG
GCCGGAAGAGAATGCTGGCCTCTGCCGGCGAACTGCAGAAGGGAAAC
GAACTGGCCCTGCCCTCCAAATATGTGAACTTCCTGTACCTGGCCAG
CCACTATGAGAAGCTGAAGGGCTCCCCCGAGGATAATGAGCAGAAAC
AGCTGTTTGTGGAACAGCACAAGCACTACCTGGACGAGATCATCGAG
CAGATCAGCGAGTTCTCCAAGAGAGTGATCCTGGCCGACGCTAATCT
GGACAAAGTGCTGTCCGCCTACAACAAGCACCGGGATAAGCCCATCA
GAGAGCAGGCCGAGAATATCATCCACCTGTTTACCCTGACCAATCTG
GGAGCCCCTGCCGCCTTCAAGTACTTTGACACCACCATCGACCGGAA
GAGGTACACCAGCACCAAAGAGGTGCTGGACGCCACCCTGATCCACC
AGAGCATCACCGGCCTGTACGAGACACGGATCGACCTGTCTCAGCTG
GGAGGCGACAAGCGTCCTGCTGCTACTAAGAAAGCTGGTCAAGCTAA

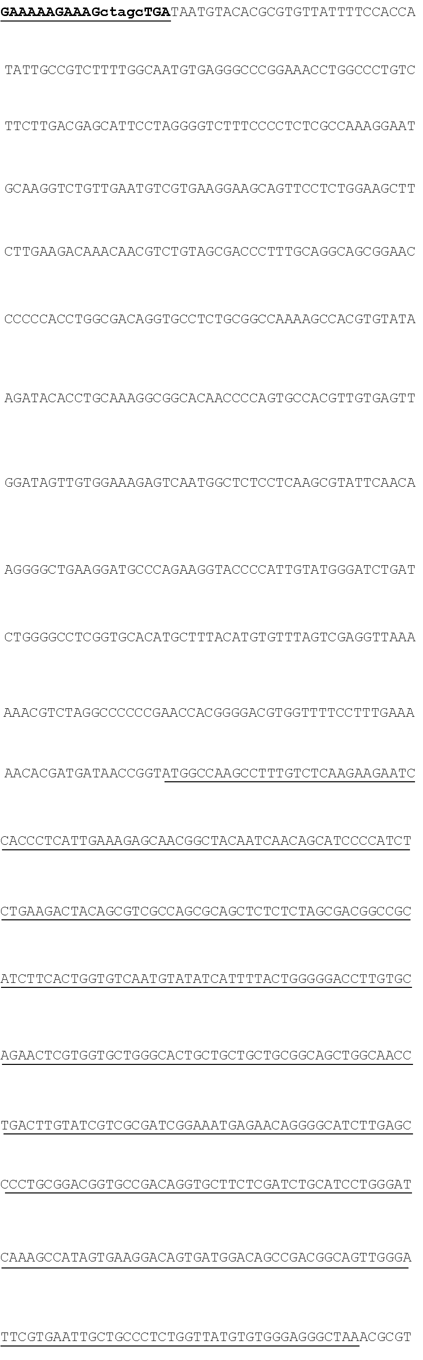

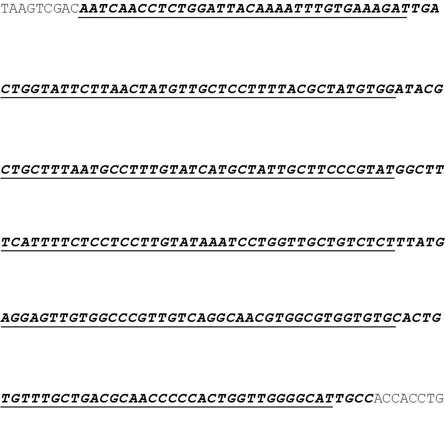

TCAGCTCCTTTCCGGGACTTTCGCTTTCCCCCTCCCTATTGCCACGG
CGGAACTCATCGCCGCCTGCCTTGCCCGCTGCTGGACAGGGGCTCGG
CTGTTGGGCACTGACAATTCCGTGGTGTTGTCGGGGAAATCATCGTC
CTTTCCTTGGCTGCTCGCCTGTGTTGCCACCTGGATTCTGCGCGGGA
CGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCT
TCGCCCTCAGACGAGTCGGATCTCCCTTTGGGCCGCCTCCCCGCGTC
GACTTTAAGACCAATGACTTACAAGGCAGCTGTAGATCTTAGCCACT

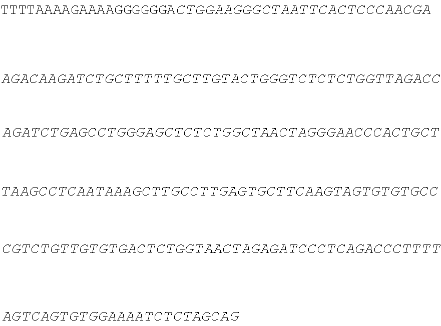

1. A CRISPR/CAS9 Self-Limiting Cas9 circuitry for Enhanced Safety (SLiCES) plasmid comprising:
an expression cassette for a Cas9 molecule;
a nucleotide sequence that encodes for a sgRNA targeting the Cas9 molecule (anti-Cas9 sgRNA); and
a nucleotide sequence that encodes for sgRNA targeting a chosen genomic locus (target sgRNA);
wherein
at least one intron is present into the open reading frame (ORF) of the expression cassette for said Cas9 molecule to form an expression cassette divided in two or more exons, and/or at least one intron is present into the nucleotides sequence encoding for the mature transcript of said anti-Cas9 sgRNA being said intron into the transcribed sequence encoding an expression cassette divided in two or more exons; and/or
the expression cassette for the Cas9 molecule and/or the sequence encoding for anti-cas9 sgRNA is preceded by a sequence including an inducible promoter.

2. The plasmid according to claim 1, wherein the anti-Cas9 sgRNA is encoded by a sequence of 17-23 nucleotide preferably.

3. The plasmid according to claim 2, wherein anti-Cas9 sgRNA encoding sequence is a sequence having at least a 60% homology with a sequence selected in the group consisting of SEQ ID N. 1-6.

4. The plasmid according to claim 1, wherein
the expression cassette for a Cas9 molecule and/or the nucleotide sequence that encodes for an anti-Cas9 sgRNA comprises at least an intron; and
the expression cassette for the Cas9 molecule and/or the sequence encoding for anti-cas9 sgRNA is preceded by a sequence including an inducible promoter.

5. The plasmid according to claim 1, wherein the expression cassette for the Cas9 molecule and the sequence encoding for anti-cas9 sgRNA are both preceded by a sequence including an inducible promoter.

6. A genetically-modified micro-organism comprising the plasmid according to claim 1.

7. A cell transfected with the plasmid according to claim 1.

8. A viral or artificial delivery system comprising the plasmid according to claim 1.

9. (canceled)

10. A method of treating a monogenic disorder, high cholesterol, antitrypsin deficiency, cancer, diabetes, infective bacterial disease or viral disease comprising administering the plasmid according to claim 1 to a subject in need thereof.

11. (canceled)

12. A pharmaceutical composition comprising the plasmid according to claim 1 and at least another pharmaceutically acceptable ingredient.

13. A process for preparing the viral system according to claim 8, the process comprising
transforming a bacterium with the plasmid according to claim 1, said bacterium wherein the expression of Cas9 and/or sgRNA is prevented by the presence of the intron or by the expression of a repressor specific for the inducible promoter or by another system apt to prevent Cas9 and/or sgRNA expression; and/or
transfecting a cell with the plasmid, said cell expressing a repressor specific for the inducible promoter or said cell comprising another system for regulating Cas9 and/or anti-Cas9 gRNA expression.

14. A method for preventing the mature expression of a toxic transcript in a bacterium, said method comprising introducing at least one intron in the nucleotide sequence encoding for said toxic transcript; being said intron into the transcribed sequence encoding an expression cassette divided in two or more exons.

15. The method according to claim 14, where the toxic transcript functions as a guide RNA, or part of it, for a nuclease.

16. (canceled)

17. A method for detecting Cas9 off-targets in in vitro cultured cells or in in vivo animal models, said method comprising introducing the plasmid according to claim 1 into the in vitro cultured cells or in vivo animal models, wherein the plasmid is introduced directly or in the form of a non-integrating vector.

18. The plasmid according to claim 1, wherein the anti-Cas9 sgRNA is encoded by a sequence of 17-23 nucleotides starting with G.

19. A pharmaceutical composition comprising the viral or artificial system according to claim 8 and at least another pharmaceutically acceptable ingredient.

20. A method of treating a monogenic disorder, high cholesterol, antitrypsin deficiency, cancer, diabetes, infective bacterial disease or viral disease comprising administering the viral or artificial system according to claim 8 to a subject in need thereof.


SEQUENCE LISTING (68 sequences)

SEQ ID NO: 1 - DNA, 21 bp, Artificial Sequence
  Description of Artificial Sequence Synthetic oligonucleotide
gtacgccggctacattgacgg

SEQ ID NO: 2 - DNA, 20 bp, Artificial Sequence
  Description of Artificial Sequence Synthetic oligonucleotide
gatccttgtagtctccgtcg

SEQ ID NO: 3 - DNA, 20 bp, Artificial Sequence
  Description of Artificial Sequence Synthetic oligonucleotide
ggctacgccggctacattga

SEQ ID NO: 4 - DNA, 20 bp, Artificial Sequence
  Description of Artificial Sequence Synthetic oligonucleotide
ggcagaaggctgacccggcg

SEQ ID NO: 5 - DNA, 20 bp, Artificial Sequence
  Description of Artificial Sequence Synthetic oligonucleotide
ggcctacagaagcgaggccc

SEQ ID NO: 6 - DNA, 20 bp, Artificial Sequence
  Description of Artificial Sequence Synthetic oligonucleotide
gagactaacgaggacgacga

SEQ ID NO: 7 - DNA, 82 bp, Artificial Sequence
  Description of Artificial Sequence Synthetic oligonucleotide
gttttagagctagaaatagcaagttaaaataaggctagtccgttatcaacttgaaaaagtggcaccgagtcggtgctttttt

SEQ ID NO: 8 - DNA, 92 bp, Artificial Sequence
  Description of Artificial Sequence Synthetic oligonucleotide
gtttaagagctatgctggaaacagcatagcaagtttaaataaggctagtccgttatcaacttgaaaaagtggcaccgagtcggtgctttttt

SEQ ID NO: 9 - DNA, 82 bp, Mus sp.
  chr12115758289-115758370
gtaaggggctcacagtagcaggcttgaggtctggacatatatatgggtgacaatgacatccactttgcctttctctccacag

SEQ ID NO: 10 - DNA, 11199 bp, Artificial Sequence
  Description of Artificial Sequence Synthetic polynucleotide
  misc_feature (1)..(255): 5′LTR
  misc_feature (256)..(1716): Lentiviral packaging sequences
  misc_feature (1717)..(1965): hU6 promoter
  misc_feature (1966)..(3846): stuffer fragment
  misc_feature (3847)..(3928): gRNA backbone
  misc_feature (3929)..(3952): spacer
  misc_feature (3953)..(4052): hH1 TetO promoter
  misc_feature (4053)..(4073): Cas-a spacer
  misc_feature (4074)..(4165): optimized gRNA backbone
  misc_feature (4166)..(4372): spacer sequence
  misc_feature (4373)..(5043): CMV TetO promoter
  misc_feature (5044)..(5055): Kozak sequence
  misc_feature (5056)..(9373): CDS for FLAG-NLS-SpCas9-NLS
  misc_feature (7267)..(7348): Intron
  misc_feature (9374)..(9387): spacer sequence
  misc_feature (9388)..(9874): ECMV-IRES
  misc_feature (9875)..(9890): spacer sequence
  misc_feature (9891)..(10289): blasticidin resistance gene
  misc_feature (10290)..(10304): spacer sequence
  misc_feature (10305)..(10559): WPRE
  misc_feature (10560)..(10963): lentiviral sequences
  misc_feature (10964)..(11199): 3′LTR-SIN
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgttttagagctagaaatagcaagttaaaataaggctagtccgttatcaacttgaaaaagtggcaccgagtcggtgcttttttgaattctagtagaattgaggtaccaatatttgcatgtcgctatgtgttctgggaaatcaccataaacgtgaaatccctatcagtgatagagacttataagttccctatcagtgatagagacaccgtacgccggctacattgacgggtttaagagctatgctggaaacagcatagcaagtttaaataaggctagtccgttatcaacttgaaaaagtggcaccgagtcggtgcttttttcaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagaggatccgccaccatggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtaaggggctcacagtagcaggcttgaggtctggacatatatatgggtgacaatgacatccactttgcctttctctccacaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaagctagctgataatgtacacgcgtgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataaccggtatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 11 - DNA, 21 bp, Artificial Sequence
  Description of Artificial Sequence Synthetic oligonucleotide
gctcgtgaccaccctgaccta

SEQ ID NO: 12 - DNA, 29 bp, Artificial Sequence
  Description of Artificial Sequence Synthetic oligonucleotide
accctcgtgaccaccctgacctacggcgt

SEQ ID NO: 13 - DNA, 29 bp, Artificial Sequence
  Description of Artificial Sequence Synthetic oligonucleotide
agcctcgtgaccaccctgacctacggagt

SEQ ID NO: 14 - DNA, 21 bp, Artificial Sequence
  Description of Artificial Sequence Synthetic oligonucleotide
gctcgtgaccaccctgacctc

SEQ ID NO: 15 - DNA, 21 bp, Artificial Sequence
  Description of Artificial Sequence Synthetic oligonucleotide
gctcgtcaccaccctgacctc

SEQ ID NO: 16 - DNA, 20 bp, Homo sapiens
  chr643769717-43769736
ggtgagtgagtgtgtgcgtg

SEQ ID NO: 17 - DNA, 29 bp, Homo sapiens
  chr643769714-43769742
gtgggtgagtgagtgtgtgcgtgtggggt

SEQ ID NO: 18 - DNA, 29 bp, Homo sapiens
  chr1465102432-65102460
gtgagtgagtgagtgtgtgtgtggggggg

SEQ ID NO: 19 - DNA, 29 bp, Homo sapiens
  chr5116098959-116098987
atgtgtgggtgagtgtgtgcgtgaggaca

SEQ ID NO: 20 - DNA, 20 bp, Homo sapiens
  chr7149432159-149432178
gtgcggcaagagcttcagcc

SEQ ID NO: 21 - DNA, 29 bp, Homo sapiens
  chr1584621794-84621822
catgtgcggcaagagcttcagccggggct

SEQ ID NO: 22 - DNA, 29 bp, Homo sapiens
  chr1943914067-43914095
ggagtgtggcaagggcttcagccaggcct

SEQ ID NO: 23 - DNA, 29 bp, Homo sapiens
  chr1527849680-27849708
ttcatggggaaagagcttcagcctgggct

SEQ ID NO: 24 - DNA, 20 bp, Homo sapiens
  chr272933853-72933872
gagtccgagcagaagaagaa

SEQ ID NO: 25 - DNA, 29 bp, Homo sapiens
  chr272933850-72933878
cctgagtccgagcagaagaagaagggctc

SEQ ID NO: 26 - DNA, 29 bp, Homo sapiens
  chr1543817546-43817574
caagagtctaagcagaagaagaagagagc

SEQ ID NO: 27 - DNA, 29 bp, Homo sapiens
  chr545358956-45358984
tcagagttagagcagaagaagaaaggcat

SEQ ID NO: 28 - DNA, 21 bp, Homo sapiens
  chr272934044-72934064
ggcctccccaaagcctggcca

SEQ ID NO: 29 - DNA, 29 bp, Homo sapiens
  chr272934038-72934066
caggcctccccaaagcctggccagggagt

SEQ ID NO: 30 - DNA, 29 bp, Homo sapiens
  chr1144964901-44964929
aagacctccccatagcctggccagggagg

SEQ ID NO: 31 - DNA, 29 bp, Homo sapiens
  chr986722462-86722490
cactcctccccacagcctggccaggggaa

SEQ ID NO: 32 - DNA, 26 bp, Artificial Sequence
  Description of Artificial Sequence Synthetic oligonucleotide
ggctacgccggctacattgacggcgg

SEQ ID NO: 33 - DNA, 29 bp, Artificial Sequence
  Description of Artificial Sequence Synthetic oligonucleotide
catgatccttgtagtctccgtcgtggtcc

SEQ ID NO: 34 - DNA, 29 bp, Artificial Sequence
  Description of Artificial Sequence Synthetic oligonucleotide
aacggctacgccggctacattgacggcgg

SEQ ID NO: 35 - DNA, 18 bp, Artificial Sequence
  Description of Artificial Sequence Synthetic oligonucleotide
gggtcttcgagaagacct

SEQ ID NO: 36 - DNA, 20 bp, Homo sapiens
  chr1955115771-55115790
gtcccctccaccccacagtg

SEQ ID NO: 37 - DNA, 33 bp, Artificial Sequence
  Description of Artificial Sequence Synthetic oligonucleotide
agagtcccctccaccccacagtgcaagaaatcc

SEQ ID NO: 38 - DNA, 33 bp, Artificial Sequence
  Description of Artificial Sequence Synthetic oligonucleotide
agagtcccctccacccaacagtgcaagaaatcc

SEQ ID NO: 39 - DNA, 33 bp, Artificial Sequence
  Description of Artificial Sequence Synthetic oligonucleotide
cagggcagaaggctgacccggcggaagaaacac

SEQ ID NO: 40 - DNA, 32 bp, Artificial Sequence
  Description of Artificial Sequence Synthetic oligonucleotide
agcgcctacagaagcgaggccctgagaatcct

SEQ ID NO: 41 - DNA, 33 bp, Artificial Sequence
  Description of Artificial Sequence Synthetic oligonucleotide
cgcgagactaacgaggacgacgagaagaaagcc

SEQ ID NO: 42 - DNA, 19 bp, Artificial Sequence
  Description of Artificial Sequence Synthetic oligonucleotide
gagacgattaatgcgtctc

SEQ ID NO: 43 - DNA, 23 bp, Artificial Sequence
  Description of Artificial Sequence Synthetic oligonucleotide
accatggtgagcaagggcgagga

SEQ ID NO: 44 - DNA, 24 bp, Artificial Sequence
  Description of Artificial Sequence Synthetic oligonucleotide
agctcgtccatgccgagagtgatc

SEQ ID NO: 45 - DNA, 22 bp, Homo sapiens
  chr643769231-43769252
gcatacgtgggctccaacaggt

SEQ ID NO: 46 - DNA, 22 bp, Homo sapiens
  chr643770011-43770032
ccgcaatgaaggggaagctcga

SEQ ID NO: 47 - DNA, 21 bp, Homo sapiens
  chr1465102697-65102717
caggcgccttgggctccgtca

SEQ ID NO: 48 - DNA, 20 bp, Homo sapiens
  chr1465101890-65101909
ccccaggatccgcgggtcac

SEQ ID NO: 49 - DNA, 23 bp, Homo sapiens
  chr5116098400-116098422
agtcagccctctgtatccctgga

SEQ ID NO: 50 - DNA, 25 bp, Homo sapiens
  chr5116099402-116099426
gagatatctgcaccctcatgttcac

SEQ ID NO: 51 - DNA, 21 bp, Homo sapiens
  chr1584621375-84621395
gactgtgggcagaggttcagc

SEQ ID NO: 52 - DNA, 24 bp, Homo sapiens
  chr1584622203-84622226
tgtatacgggacttgactcagacc

SEQ ID NO: 53 - DNA, 21 bp, Homo sapiens
  chr1943914466-43914486
cacgactgcaggctcatgagc

SEQ ID NO: 54 - DNA, 23 bp, Homo sapiens
  chr1943913657-43913679
gaagcgcttaccacacacatcac

SEQ ID NO: 55 - DNA, 23 bp, Homo sapiens
  chr1527849277-27849299
agtcacatgctgcctggattgac

SEQ ID NO: 56 - DNA, 24 bp, Homo sapiens
  chr1527850093-27850116
gtggaggagatttctctaggagag

SEQ ID NO: 57 - DNA, 23 bp, Homo sapiens
  chr272933595-72933617
ctgccatccccttctgtgaatgt

SEQ ID NO: 58 - DNA, 22 bp, Homo sapiens
  chr272934372-72934393
ggaatctaccaccccaggctct

SEQ ID NO: 59 - DNA, 24 bp, Homo sapiens
  chr1543816987-43817010
ctgctgtttcctgaagctgccact

SEQ ID NO: 60 - DNA, 25 bp, Homo sapiens
  chr1543817874-43817898
ctgccatggaaattccagagggaac

SEQ ID NO: 61 - DNA, 24 bp, Homo sapiens
  chr545358743-45358766
tgtggggagatttgcatctgtgga

SEQ ID NO: 62 - DNA, 27 bp, Homo sapiens
  chr545359326-45359352
ttgagacatggggatagaatcatgaac

SEQ ID NO: 63 - DNA, 25 bp, Homo sapiens
  chr1144965382-44965406
tgaacgaatcaggtctgagaggatc

SEQ ID NO: 64 - DNA, 23 bp, Homo sapiens
  chr1144964517-44964539
gagcttcactccagagaggctgt

SEQ ID NO: 65 - DNA, 23 bp, Homo sapiens
  chr986722951-86722973
tgctactgctggctgcagagatg

SEQ ID NO: 66 - DNA, 24 bp, Homo sapiens
  chr986722025-86722048
gcattcgttttgggaggcagagga

SEQ ID NO: 67 - DNA, 842 bp, Artificial Sequence
  Description of Artificial Sequence Synthetic polynucleotide
atgggcaagcctatccccaaccctctcctcggtctcgattctacggctagcctcgtgaccaccctgacctccggagtgtaggaggaggaggaggcagcggcggaggcggaagcgggcggccgctagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccacaggaaaactccctgtcccttggccaactctggtcactacacttacatacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaataa

SEQ ID NO: 68 - DNA, 615 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccacaggaaaactccctgtcccttggccaactctggtcactacacttacatacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcaagtaa